SEQUENCE LISTING (13 sequences)

SEQ ID NO: 1 - DNA, 1350 bp, Mus musculus
gtgaatgcgactcgctcatcgtccgcgctccccgcccgcccgcccgccgggacgtggtaggggatgcctagctcctctgcgatggcagttggcgcgctctccagttccctcttggtcacctgctgcctgatggtggctctgtgcagtccgagcatcccgctggagaagctggcccaagctcctgagcaacccggccaggagaagcgcgagcacgcgtctcgggacagcccggggcgggtgagcgagctcgggcgcgcctcgagggacgagggcagcagtgcccgggactggaagagcaagggcagccgcgcgctctcgggccgggaggcgtggagcaagcagaagcaggcctgggctgcccagggcgggagcgccaaggccgcggactggcaggtccggcctcgcggggataccccacagggggagcccccggccgccgcccaggaagctatcagcctggaactcgttcccacaccggagctgcctgaggaatacgcctacccggactaccgcgggaagggctgtgtggacgagagtggcttcgtgtatgcgatcggggagaagttcgccccaggtccctcagcctgcccgtgtctgtgtacggaggaggggcccctttgcgcacagccagagtgcccacggcttcacccgcgctgcatccacgtcgacaacagccagtgctgtccgcagtgcaaggagaagaagaactactgcgagttccggggcaagacctaccagactttggaggagtttgtggtgtctccatgtgaacggtgccgctgtgaagccaatggtgaagtgctgtgcacagtgtcggcgtgtccccagacggaatgtgtggaccccgtatatgagcctgatcagtgctgcccgatctgcaaaaacggtccaaactgctttgcagaaaccgctgtaatcccagctggtagagaggtgaagactgacgagtgcaccatatgtcactgcacttatgaagagggcacgtggagaattgaacggcaagccatgtgcacgagacatgagtgcagacaaatgtagacttcacaatacagaaactctcatgtttttctagaatattttactaatgtgacattctagatgactctgagaaatatcaatcaaaaacagaagactttggataaggagtcaaggaaaatggttggtacttttgtttttcttggtaacaaatacagcaagagacagaaatggatgtatttcaaaacatcaataagaactttgggcatactccctctctaaataaatgtgctattttcacaactttattttaggaatgagtgtatctgcatatctccatgcttctgtgtaatacaactgagcagtgcagcggcg

SEQ ID NO: 2 - protein, 324 aa, Mus musculus
MPSSSAMAVGALSSSLLVTCCLMVALCSPSIPLEKLAQAPEQPGQEKREHASRDSPGRVSELGRASRDEGSSARDWKSKGSRALSGREAWSKQKQAWAAQGGSAKAADWQVRPRGDTPQGEPPAAAQEAISLELVPTPELPEEYAYPDYRGKGCVDESGFVYAIGEKFAPGPSACPCLCTEEGPLCAQPECPRLHPRCIHVDNSQCCPQCKEKKNYCEFRGKTYQTLEEFVVSPCERCRCEANGEVLCTVSACPQTECVDPVYEPDQCCPICKNGPNCFAETAVIPAGREVKTDECTICHCTYEEGTWRIERQAMCTRHECRQM

SEQ ID NO: 3 - DNA, 1013 bp, Mus musculus
gcattcccatggaaggagctgagtgtgtttaatattaggagaacatccagaagtctgtgaggagggggatggctcttcatattcatgaagcttgcatacttctattggtcatccctggattggtcacctctgctgccatcagtcacgaagactatcctgctgatgaaggtgaccaggcttccagtaatgacaatctgatctttgatgactatcgagggaaagggtgtgtggatgacagcggctttgtatacaagttgggagaacggtttttcccggggcattccaactgtccgtgtgtctgtgccctagatggacctgtctgtgaccagccagaatgccctaaaattcatccgaaatgtacaaaggtggaacacaatggatgctgtcccgaatgtaaagaagtgaaaaacttttgtgaatatcatgggaaaaattacaaaatcttggaggaatttaagccctctccatgtgaatggtgtcgctgtgagcccagcaatgaagttcactgtgttgtagcagactgcgcagttcctgagtgtgtcaacccaatctatgaaccagagcaatgttgtcctgtctgcaaaaatggtccaaactgctttgcaggaactacaataattccagctggcattgaagtgaaagtggacgactgtaacatctgtcactgtcacaacggggactggtggaaacctgctcagtgttcaaagcgcgaatgccaaggcaagcagactgtgtagggtagattcccgaccagtgacttgttctaggaggggatgctatggcttctacactgcacatgtatcgcgctcaacagaatgagcaaaccaacccctgccagctacaacagggtcaccagcaaaaccttctggggttgacaaaagtgaatattttacgaagaggaaatctctctctctttctcttgctatataaaatatacatagcatatagctatctacaccacttttgtaattgccaatgcttgatggtaactagacaaccaagctttcttgggc

SEQ ID NO: 4 - protein, 222 aa, Mus musculus
MALHIHEACILLLVIPGLVTSAAISHEDYPADEGDQASSNDNLIFDDYRGKGCVDDSGFVYKLGERFFPGHSNCPCVCALDGPVCDQPECPKIHPKCTKVEHNGCCPECKEVKNFCEYHGKNYKILEEFKPSPCEWCRCEPSNEVHCVVADCAVPECVNPIYEPEQCCPVCKNGPNCFAGTTIIPAGIEVKVDDCNICHCHNGDWWKPAQCSKRECQGKQTV

SEQ ID NO: 5 - DNA, 423 bp, Mus musculus
gggatggctcttcatattcatgaagcttgcatacttctattggtcatccctggattggtcacctctgctgccatcagtcacgaagactatcctgctgatgaaggtgaccaggcttccagtaatgacaatctgatctttgatgactatcgagggaaagggtgtgtggatgacagcggctttgtatacaagttgggagaacggtttttcccggggcattccaactgtccgtgtgtctgtgccctagatggacctgtctgtgaccagccagaatgccctaaaattcatccgaaatgtacaaaggtggaacacaatggatgctgtcccgaatgtaaagaagtgaaaaacttttgtgaatatcatgggaaaaattacaaaatcttggaggaatttaaggtccaaactgctttgcaggaactacaataa

SEQ ID NO: 6 - protein, 139 aa, Mus musculus
MALHIHEACILLLVIPGLVTSAAISHEDYPADEGDQASSNDNLIFDDYRGKGCVDDSGFVYKLGERFFPGHSNCPCVCALDGPVCDQPECPKIHPKCTKVEHNGCCPECKEVKNFCEYHGKNYKILEEFKVQTALQELQ

SEQ ID NO: 7 - DNA, 282 bp, Mus musculus
gggatggctcttcatattcatgaagcttgcatacttctattggtcatccctggattggtcacctctgctgccatcagtcacgaagactatcctgctgatgaagatggacctgtctgtgaccagccagaatgccctaaaattcatccgaaatgtacaaaggtggaacacaatggatgctgtcccgaatgtaaagaagtgaaaaacttttgtgaatatcatgggaaaaattacaaaatcttggaggaatttaaggtccaaactgctttgcaggaactacaataa

SEQ ID NO: 8 - protein, 92 aa, Mus musculus
MALHIHEACILLLVIPGLVTSAAISHEDYPADEDGPVCDQPECPKIHPKCTKVEHNGCCPECKEVKNFCEYHGKNYKILEEFKVQTALQELQ

SEQ ID NO: 9 - DNA, 704 bp, Homo sapiens
gggatggctcttcatattcatgaagcttgcatacttctgttggtcatccctggattggtcacctctgctgctatcagtcatgaagactatcctgctgatgaaggtgaccagatctccagtaatgacaatctgatctttgatgactatcgagggaaagggtgtgtcgatgacagcggctttgtatacaagttgggagaacgatttttccctgggcattccaactgtccatgtgtctgtgctctagatggacctgtttgcgaccaaccagaatgccctaaaattcacccaaagtgtactaaagtggaacacaatggatgctgtcctgagtgcaaagaagtaaaaaacttctgtgaatatcacgggaaaaattacaaaatcttggaggaatttaagccctctccatgtgaatggtgtcgctgtgagcccagcaatgaagttcactgtgttgtagcagactgcgcagttcctgagtgtgtcaacccagtctatgaaccagaacaatgttgtcctgtctgcaaaaatggtccaaactgctttgcaggaacgacgataattccagctggcattgaagtgaaagtggacgaatgtaacatctgtcattgtcacaacggggactggtggaagcctgctcagtgttcgaaacgtgaatgccaaggcaagcagactgtgtaggacaaacttccacccaatgatgagttcttagg

SEQ ID NO: 10 - protein, 222 aa, Homo sapiens
MALHIHEACILLLVIPGLVTSAAISHEDYPADEGDQISSNDNLIFDDYRGKGCVDDSGFVYKLGERFFPGHSNCPCVCALDGPVCDQPECPKIHPKCTKVEHNGCCPECKEVKNFCEYHGKNYKILEEFKPSPCEWCRCEPSNEVHCVVADCAVPECVNPVYEPEQCCPVCKNGPNCFAGTTIIPAGIEVKVDECNICHCHNGDWWKPAQCSKRECQGKQTV

SEQ ID NO: 11 - DNA, 539 bp, Homo sapiens
gggatggctcttcatattcatgaagcttgcatacttctgttggtcatccctggattggtcacctctgctgctatcagtcatgaagactatcctgctgatgaaggtgaccagatctccagtaatgacaatctgatctttgatgactatcgagggaaagggtgtgtcgatgacagcggctttgtatacaagttgggagaacgatttttccctgggcattccaactgtccatgtgtctgtgctctagatggacctgtttgcgaccaaccagaatgccctaaaattcacccaaagtgtactaaagtggaacacaatggatgctgtcctgagtgcaaagaagtaaaaaacttctgtgaatatcgcgggaaaaattacaaaatcttggaggaatttaaggtccaaactgctttgcaggaacgacgataattccagctggcattgaagtgaaagtggacgaatgtaacatctgtcattgtcacaacggggactggtggaagcctgctcagtgttcgaaacgtgaatgccaaggcaagcagactgta

SEQ ID NO: 12 - protein, 139 aa, Homo sapiens [sequence omitted]

SEQ ID NO: 13 - protein, 16 aa, Artificial
  Synthetic peptide for generating G11 antibody
EHASRDSPGRVSELGR

(19) United States
(12) Patent Application Publication
(10) Pub. No.: US 2009/0258023 A1
(43) Pub. Date: Oct. 15, 2009

(54) TGF-BETA SUPERFAMILY BINDING PROTEINS AND MODULATION OF BONE FORMATION AND LOSS

(75) Inventors: Yoshiyuki Mochida, Chapel Hill, NC (US); Mitsuo Yamauchi, Chapel Hill, NC (US)

Correspondence Address: MYERS BIGEL SIBLEY & SAJOVEC, PO BOX 37428, RALEIGH, NC, 27627, US

(73) Assignee: University of North Carolina at Chapel Hill

(21) Appl. No.: 12/421,082

(22) Filed: Apr. 9, 2009

Related U.S. Application Data
(60) Provisional application No. 61/043,873, filed on Apr. 10, 2008.

(51) Int. Cl. A61K 39/395; C12N 15/11; C12N 15/00; C12N 5/08; C07K 14/00; C12P 21/02; C07K 16/18; A61K 31/7088; A61K 38/16; C12N 5/06; G01N 33/566; G01N 33/53

(52) U.S. Cl. 424/139.1; 536/23.5; 435/320.1; 435/366; 530/350; 536/23.4; 435/69.1; 530/387.9; 536/24.5; 514/44 A; 514/12; 435/375; 436/501; 435/7.21

(57) ABSTRACT

The present invention relates to novel polynucleotides and polypeptides that are expressed in osteoblasts, interact with members of the TGF-β superfamily, and inhibit mineralization. The invention further relates to the use of these polynucleotides and polypeptides to modulate bone formation and bone loss.

### STATEMENT OF PRIORITY

This application claims the benefit, under 35 U.S.C. §119(e), of U.S. Provisional Application Ser. No. 61/043,873, filed Apr. 10, 2008, the entire contents of which are incorporated by reference herein.

### STATEMENT OF FEDERAL SUPPORT

This invention was made, in part, with government support under grant numbers DE10489 and AR052824 from the National Institutes of Health. The United States government has certain rights to this invention.

### FIELD OF THE INVENTION

The present invention relates to novel polynucleotides and polypeptides that are expressed in osteoblasts, interact with members of the transforming growth factor-beta (TGF-β) superfamily, and inhibit mineralization. The invention further relates to the use of these polynucleotides and polypeptides to modulate bone formation and bone loss.

### BACKGROUND OF THE INVENTION

The TGF-β superfamily consists of three major sub-families, including the TGF-β, bone morphogenetic protein (BMP) and activin/nodal sub-families. Members of the superfamily are known to be potent effectors in almost all crucial biological, developmental, and regenerative events. Currently, however, the presence of members of the different TGF-β subfamilies in osteoblasts and, if present, their roles in osteoblast function and mineralization are largely unknown.

The present invention addresses previous shortcomings in the art by providing novel polynucleotides encoding polypeptides that are expressed in osteoblasts, interact with members of the TGF-β superfamily, and inhibit mineralization.

### SUMMARY OF THE INVENTION

The present invention is based, in part, on the discovery of two new genes designated G11 and F12. The polypeptides encoded by these genes are implicated in osteoblast function and mineralization and provide an important new set of therapeutic targets.

Accordingly, as one aspect, the invention provides an isolated polynucleotide encoding a polypeptide selected from the group consisting of: (a) a polynucleotide comprising a nucleotide sequence at least 70% identical to a nucleotide sequence selected from the group consisting of SEQ ID NOS:1, 3, 5, 7, 9, and 11, and encoding a functional polypeptide; (b) a polynucleotide that hybridizes to a nucleotide sequence selected from the group consisting of SEQ ID NOS:1, 3, 5, 7, 9, and 11 under stringent hybridization conditions and encodes a functional polypeptide; (c) a polynucleotide encoding a functional polypeptide comprising an amino acid sequence at least 70% identical to an amino acid sequence selected from the group consisting of SEQ ID NOS:2, 4, 6, 8, 10, and 12; and (d) a functional fragment of any of (a) to (c). Also provided are isolated polynucleotides encoding a functional fragment of G11 or F12.

As a further aspect, the invention provides polypeptides encoded by the isolated polynucleotide sequences. Further provided are cells comprising the isolated polynucleotides and polypeptides of the invention. In another aspect, the invention relates to recombinant methods of producing the polypeptides of the invention.

As yet another aspect, the invention provides an antibody that specifically binds to the polypeptides of the invention.

As yet another aspect, the invention provides an antisense oligonucleotide or a siRNA that specifically binds to the polynucleotides of the invention. In one embodiment, the antisense oligonucleotide or siRNA inhibits the transcription and/or translation of the polynucleotides of the invention.

In one aspect, the invention relates to pharmaceutical compositions comprising the polynucleotides, polypeptides, or antibodies of the invention and a pharmaceutically acceptable carrier.

As still another aspect, the invention provides a method of inhibiting mineralization in a cell, comprising delivering to said cell a polynucleotide or polypeptide of the invention. The invention further provides a method of inhibiting bone formation in a subject, comprising delivering to said subject a polynucleotide or polypeptide of the invention. The invention also provides a method of inhibiting bone loss in a subject, comprising delivering to said subject a compound that decreases the activity of the polypeptide of the invention in an amount effective to decrease the activity of the polypeptide in the cell. In particular embodiments, the compound is an antisense oligonucleotide, an siRNA, or an antibody. In other representative embodiments, the compound is an antisense oligonucleotide or siRNA that is targeted against the G11 or F12 polynucleotide. In other embodiments, the compound is an antibody that binds to the G11 or F12 polypeptide. The methods can be carried out in cultured cells or in vivo.

The present invention further provides screening methods using the polynucleotides and polypeptides of the invention as targets. The screening methods can be carried out in cell-free assays, in cultured cells, or in live organisms, such as transgenic non-human animals, plants, fungi, or bacteria.

As one particular aspect, the invention provides a method for identifying a compound that binds to a G11 or F12 polypeptide or a functional fragment thereof, comprising: contacting the polypeptide with a test compound under conditions whereby binding between the polypeptide and the test compound can be detected; and detecting binding between the polypeptide and the test compound.

As another aspect, the invention provides a method of identifying a compound that modulates the activity of a G11 or F12 polypeptide or a functional fragment thereof, comprising: contacting the polypeptide with a test compound under conditions whereby modulation of the activity of the polypeptide can be detected; and detecting modulation of the activity of the polypeptide.

As still a further aspect, the invention provides a method of identifying a compound that can modulate mineralization, comprising: contacting a G11 or F12 polypeptide or a functional fragment thereof with a test compound under conditions whereby modulation of the activity of the polypeptide can be detected; and detecting modulation of the activity of the polypeptide, thereby identifying a compound that can modulate mineralization.

These and other aspects of the invention are set forth in more detail in the description of the invention below.

### BRIEF DESCRIPTION OF THE DRAWINGS

FIGS. 1A-1D show a schematic representation of the mouse G11 and F12 gene/protein. FIG. 1A shows the mouse G11 protein structure. FIG. 1B shows the mouse F12 protein structure. FIG. 1C shows the exon-intron structure of the mouse G11 gene. FIG. 1D shows the phylogenetic tree analysis of G11 and F12 together with other mouse cysteine knot protein members. Clustal X and W were used.

FIGS. 2A-2B depict the G11 and F12 gene expression pattern in MC3T3-E1 cells during the course of cell culture. The expression was determined by quantitative real time PCR analysis. FIG. 2A depicts G11 expression. FIG. 2B depicts F12 expression.

FIG. 3 shows the characterization of anti-G11 antibody. Equal concentration of bovine serum albumin (BSA) and purified G11-V5/His protein (G11) were subjected to Western blot analysis using anti-G11, anti-V5 antibodies and preimmune rabbit serum.

FIGS. 4A-4F show the characterization of MC3T3-E1 cell derived clones expressing G11-V5 F12-V5/His fusion protein. FIG. 4A shows immunoprecipitation/Western blot analysis with anti-V5 antibody for the clones (G1, G2 and G3) overexpressing G11-V5/His protein and controls (MC and EV). FIG. 4B shows immunoprecipitation/Western blot analysis with anti-V5 antibody for the clones (F1, F2 and F3) overexpressing F12-V5/His protein and controls (MC and EV). FIG. 4C shows the results of an in vitro mineralization assay in G clones compared to MC and EV controls. FIG. 4D shows the results of an in vitro mineralization assay in F clones compared to MC and EV controls. FIG. 4E shows the results of a cell proliferation assay in G3 clone compared to controls. FIG. 4F shows the effect of anti-G1 antibody on in vitro mineralization. MC; MC3T3-E1 cells, EV; clone transfected with an empty vector, NRI; normal rabbit immunoglobulin.

FIG. 5A shows the binding of G11 to TGF-β superfamily members. BMP-2, -4, -6, -7, TGF-β1, -β1-LAP, -β2, -β3, inhibin-ba, -bb, and nodal were subcloned into pcDNA3.1-V5/His vectors. pcDNA3-G11 fused with HA tag was transfected together with TGF-β superfamily members into 293 cells and cultured medium was collected. Immunoprecipitation/Western blot analyses were performed. L; TGF-beta1 LAP, Inh; Inhibin. FIG. 5B shows the binding of F12 to TGF-β superfamily members. BMP-2, -4, -6, -7, TGF-β1, -β1-LAP, -β2, -β3, inhibin-ba, -bb, and nodal were subcloned into pcDNA3.1-V5/His vectors. pcDNA3-F12 fused with HA tag was transfected together with TGF-β superfamily members into 293 cells and cultured medium was collected. Immunoprecipitation/Western blot analyses were performed. L; TGF-beta1 LAP, Inh; Inhibin. FIG. 5C shows the effect of G11 protein on activin/nodal signaling.

FIG. 6 shows the polynucleotide and amino acid sequences for mouse G11 (SEQ ID NOS:1 and 2).

FIG. 7 shows the polynucleotide and amino acid sequences for mouse F12-1 (SEQ ID NOS:3 and 4).

FIG. 8 shows the polynucleotide and amino acid sequences for mouse F12-2 (SEQ ID NOS:5 and 6).

FIG. 9 shows the polynucleotide and amino acid sequences for mouse F12-3 (SEQ ID NOS:7 and 8).

FIG. 10 shows the polynucleotide and amino acid sequences for human F12-1 (SEQ ID NOS:9 and 10).

FIG. 11 shows the polynucleotide and amino acid sequences for human F12-2 (SEQ ID NOS:11 and 12).

### DETAILED DESCRIPTION OF THE INVENTION

The present invention will now be described in more detail with reference to the accompanying drawings, in which preferred embodiments of the invention are shown. This invention may, however, be embodied in different forms and should not be construed as limited to the embodiments set forth herein. Rather, these embodiments are provided so that this disclosure will be thorough and complete, and will fully convey the scope of the invention to those skilled in the art.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention. All publications, patent applications, patents, patent publications and other references cited herein are incorporated by reference in their entireties for the teachings relevant to the sentence and/or paragraph in which the reference is presented.

Nucleotide sequences are presented herein by single strand only, in the 5′ to 3′ direction, from left to right, unless specifically indicated otherwise. Nucleotides and amino acids are represented herein in the manner recommended by the IUPAC-IUB Biochemical Nomenclature Commission, or (for amino acids) by either the one-letter code, or the three letter code, both in accordance with 37 C.F.R. §1.822 and established usage.

Except as otherwise indicated, standard methods known to those skilled in the art may be used for cloning genes, amplifying and detecting nucleic acids, and the like. Such techniques are known to those skilled in the art. See, e.g., Sambrook et al., Molecular Cloning: A Laboratory Manual 2nd Ed. (Cold Spring Harbor, N.Y., 1989); Ausubel et al. Current Protocols in Molecular Biology (Green Publishing Associates, Inc. and John Wiley & Sons, Inc., New York).

### I. Definitions

As used in the description of the invention and the appended claims, the singular forms “a,” “an,” and “the” are intended to include the plural forms as well, unless the context clearly indicates otherwise.

Also as used herein, “and/or” refers to and encompasses any and all possible combinations of one or more of the associated listed items, as well as the lack of combinations when interpreted in the alternative (“or”).

The term “consists essentially of” (and grammatical variants), as applied to a polynucleotide or polypeptide sequence of this invention, means a polynucleotide or polypeptide that consists of both the recited sequence (e.g., SEQ ID NO) and a total of ten or less (e.g., 1, 2, 3, 4, 5, 6, 7, 8, 9, or 10) additional nucleotides or amino acids on the 5′ and/or 3′ or N-terminal and/or C-terminal ends of the recited sequence such that the function of the polynucleotide or polypeptide is not materially altered. The total of ten or less additional nucleotides or amino acids includes the total number of additional nucleotides or amino acids on both ends added together. The term “materially altered,” as applied to polynucleotides of the invention, refers to an increase or decrease in ability to express the encoded polypeptide of at least about 50% or more as compared to the expression level of a polynucleotide consisting of the recited sequence. The term “materially altered,” as applied to polypeptides of the invention, refers to an increase or decrease in binding affinity to one or more members of the TGF-β superfamily of at least about 50% or more as compared to the binding affinity of a polypeptide consisting of the recited sequence.

The term “modulate,” “modulates,” or “modulation” refers to enhancement (e.g., an increase) or inhibition (e.g., a reduction) in the specified activity.

The term “enhance,” “enhances,” “enhancing,” or “enhancement” refers to an increase in the specified parameter (e.g., at least about a 1.25-fold, 1.5-fold, 2-fold, 3-fold, 4-fold, 5-fold, 6-fold, 8-fold, 10-fold, twelve-fold, or even fifteen-fold or more increase).

The term “inhibit” or “reduce” or grammatical variations thereof as used herein refers to a decrease or diminishment in the specified activity of at least about 25%, 35%, 40%, 50%, 60%, 75%, 80%, 90%, 95% or more. In particular embodiments, the inhibition or reduction results in little or essentially no detectible activity (at most, an insignificant amount, e.g., less than about 10% or even 5%).

A “therapeutically effective” amount as used herein is an amount that provides some improvement or benefit to the subject. Alternatively stated, a “therapeutically effective” amount is an amount that will provide some alleviation, mitigation, or decrease in at least one clinical symptom in the subject (e.g., bone loss). Those skilled in the art will appreciate that the therapeutic effects need not be complete or curative, as long as some benefit is provided to the subject.

By the terms “treat,” “treating,” or “treatment of,” it is intended that the severity of the subject's condition is reduced or at least partially improved or modified and that some alleviation, mitigation or decrease in at least one clinical symptom is achieved.

As used herein, a “vector” or “delivery vector” can be a viral or non-viral vector that is used to deliver a nucleic acid to a cell, tissue or subject.

A “recombinant” vector or delivery vector refers to a viral or non-viral vector that comprises one or more heterologous nucleotide sequences (i.e., transgenes), e.g., two, three, four, five or more heterologous nucleotide sequences.

As used herein, “nucleic acid,” “nucleotide sequence” and “polynucleotide” encompass both RNA and DNA, including cDNA, genomic DNA, mRNA, synthetic (e.g., chemically synthesized) DNA and chimeras of RNA and DNA. The term polynucleotide or nucleotide sequence refers to a chain of nucleotides without regard to length of the chain. The nucleic acid can be double-stranded or single-stranded. Where single-stranded, the nucleic acid can be a sense strand or an antisense strand. The nucleic acid can be synthesized using oligonucleotide analogs or derivatives (e.g., inosine or phosphorothioate nucleotides). Such oligonucleotides can be used, for example, to prepare nucleic acids that have altered base-pairing abilities or increased resistance to nucleases. The present invention further provides a nucleic acid that is the complement (which can be either a full complement or a partial complement) of a nucleic acid or nucleotide sequence of this invention.

An “isolated polynucleotide” is a nucleotide sequence (e.g., DNA or RNA) that is not immediately contiguous with nucleotide sequences with which it is immediately contiguous (one on the 5′ end and one on the 3′ end) in the naturally occurring genome of the organism from which it is derived. Thus, in one embodiment, an isolated nucleic acid includes some or all of the 5′ non-coding (e.g., promoter) sequences that are immediately contiguous to a coding sequence. The term therefore includes, for example, a recombinant DNA that is incorporated into a vector, into an autonomously replicating plasmid or virus, or into the genomic DNA of a prokaryote or eukaryote, or which exists as a separate molecule (e.g., a cDNA or a genomic DNA fragment produced by PCR or restriction endonuclease treatment), independent of other sequences. It also includes a recombinant DNA that is part of a hybrid nucleic acid encoding an additional polypeptide or peptide sequence.

The term “isolated” can refer to a nucleic acid, nucleotide sequence or polypeptide that is substantially free of cellular material, viral material, and/or culture medium (when produced by recombinant DNA techniques), or chemical precursors or other chemicals (when chemically synthesized). Moreover, an “isolated fragment” is a fragment of a nucleic acid, nucleotide sequence or polypeptide that is not naturally occurring as a fragment and would not be found in the natural state. “Isolated” does not mean that the preparation is technically pure (homogeneous), but it is sufficiently pure to provide the polypeptide or nucleic acid in a form in which it can be used for the intended purpose.

An isolated cell refers to a cell that is separated from other components with which it is normally associated in its natural state. For example, an isolated cell can be a cell in culture medium and/or a cell in a pharmaceutically acceptable carrier of this invention. Thus, an isolated cell can be delivered to and/or introduced into a subject. In some embodiments, an isolated cell can be a cell that is removed from a subject and manipulated as described herein ex vivo and then returned to the subject.

The term “fragment,” as applied to a polynucleotide, will be understood to mean a nucleotide sequence of reduced length relative to a reference nucleic acid or nucleotide sequence and comprising, consisting essentially of, and/or consisting of a nucleotide sequence of contiguous nucleotides identical or almost identical (e.g., 90%, 92%, 95%, 98%, 99% identical) to the reference nucleic acid or nucleotide sequence. Such a nucleic acid fragment according to the invention may be, where appropriate, included in a larger polynucleotide of which it is a constituent. In some embodiments, such fragments can comprise, consist essentially of, and/or consist of oligonucleotides having a length of at least about 8, 10, 12, 15, 20, 25, 30, 35, 40, 45, 50, 75, 100, 150, 200, or more consecutive nucleotides of a nucleic acid or nucleotide sequence according to the invention.

The term “fragment,” as applied to a polypeptide, will be understood to mean an amino acid sequence of reduced length relative to a reference polypeptide or amino acid sequence and comprising, consisting essentially of, and/or consisting of an amino acid sequence of contiguous amino acids identical or almost identical (e.g., 90%, 92%, 95%, 98%, 99% identical) to the reference polypeptide or amino acid sequence. Such a polypeptide fragment according to the invention may be, where appropriate, included in a larger polypeptide of which it is a constituent. In some embodiments, such fragments can comprise, consist essentially of, and/or consist of peptides having a length of at least about 4, 6, 8, 10, 12, 15, 20, 25, 30, 35, 40, 45, 50, 75, 100, 150, 200, or more consecutive amino acids of a polypeptide or amino acid sequence according to the invention.

A “vector” is any nucleic acid molecule for the cloning of and/or transfer of a nucleic acid into a cell. A vector may be a replicon to which another nucleotide sequence may be attached to allow for replication of the attached nucleotide sequence. A “replicon” can be any genetic element (e.g., plasmid, phage, cosmid, chromosome, viral genome) that functions as an autonomous unit of nucleic acid replication in vivo, i.e., capable of replication under its own control. The term “vector” includes both viral and nonviral nucleic acid molecules for introducing a nucleic acid into a cell in vitro, ex vivo, and/or in vivo. A large number of vectors known in the art may be used to manipulate nucleic acids, incorporate response elements and promoters into genes, etc. For example, the insertion of the nucleic acid fragments corresponding to response elements and promoters into a suitable vector can be accomplished by ligating the appropriate nucleic acid fragments into a chosen vector that has complementary cohesive termini. Alternatively, the ends of the nucleic acid molecules may be enzymatically modified or any site may be produced by ligating nucleotide sequences (linkers) to the nucleic acid termini. Such vectors may be engineered to contain sequences encoding selectable markers that provide for the selection of cells that contain the vector and/or have incorporated the nucleic acid of the vector into the cellular genome. Such markers allow identification and/or selection of host cells that incorporate and express the proteins encoded by the marker.

Viral vectors, and particularly retroviral vectors, have been used in a wide variety of gene delivery applications in cells, as well as living animal subjects. Viral vectors that can be used include, but are not limited to, retrovirus, lentivirus, adeno-associated virus, poxvirus, alphavirus, baculovirus, vaccinia virus, herpes virus, Epstein-Barr virus, adenovirus, geminivirus, and caulimovirus vectors. Non-viral vectors include plasmids, liposomes, electrically charged lipids (cytofectins), nucleic acid-protein complexes, and biopolymers. In addition to a nucleic acid of interest, a vector may also comprise one or more regulatory regions, and/or selectable markers useful in selecting, measuring, and monitoring nucleic acid transfer results (delivery to specific tissues, duration of expression, etc.).

Vectors may be introduced into the desired cells by methods known in the art, e.g., transfection, electroporation, microinjection, transduction, cell fusion, DEAE dextran, calcium phosphate precipitation, lipofection (lysosome fusion), use of a gene gun, or a nucleic acid vector transporter (see, e.g., Wu et al., J. Biol. Chem. 267:963 (1992); Wu et al., J. Biol. Chem. 263:14621 (1988); and Hartmut et al., Canadian Patent Application No. 2,012,311, filed Mar. 15, 1990).

In some embodiments, a polynucleotide of this invention can be delivered to a cell in vivo by lipofection. Synthetic cationic lipids designed to limit the difficulties and dangers encountered with liposome-mediated transfection can be used to prepare liposomes for in vivo transfection of a nucleotide sequence of this invention (Felgner et al., Proc. Natl. Acad. Sci. USA 84:7413 (1987); Mackey, et al., Proc. Natl. Acad. Sci. U.S.A. 85:8027 (1988); and Ulmer et al., Science 259:1745 (1993)). The use of cationic lipids may promote encapsulation of negatively charged nucleic acids, and also promote fusion with negatively charged cell membranes (Felgner et al., Science 337:387 (1989)). Particularly useful lipid compounds and compositions for transfer of nucleic acids are described in International Patent Publications WO95/18863 and WO96/17823, and in U.S. Pat. No. 5,459,127. The use of lipofection to introduce exogenous nucleotide sequences into specific organs in vivo has certain practical advantages. Molecular targeting of liposomes to specific cells represents one area of benefit. It is clear that directing transfection to particular cell types would be particularly preferred in a tissue with cellular heterogeneity, such as pancreas, liver, kidney, and the brain. Lipids may be chemically coupled to other molecules for the purpose of targeting (Mackey, et al., 1988, supra). Targeted peptides, e.g., hormones or neurotransmitters, and proteins such as antibodies, or non-peptide molecules can be coupled to liposomes chemically.

In various embodiments, other molecules can be used for facilitating delivery of a nucleic acid in vivo, such as a cationic oligopeptide (e.g., WO95/21931), peptides derived from nucleic acid binding proteins (e g., WO96/25508), and/or a cationic polymer (e.g., WO95/21931).

It is also possible to introduce a vector in vivo as naked nucleic acid (see U.S. Pat. Nos. 5,693,622, 5,589,466 and 5,580,859). Receptor-mediated nucleic acid delivery approaches can also be used (Curiel et al., Hum. Gene Ther. 3:147 (1992); Wu et al., J. Biol. Chem. 262:4429 (1987)).

The term “transfection” or “transduction” means the uptake of exogenous or heterologous nucleic acid (RNA and/or DNA) by a cell. A cell has been “transfected” or “transduced” with an exogenous or heterologous nucleic acid when such nucleic acid has been introduced or delivered inside the cell. A cell has been “transformed” by exogenous or heterologous nucleic acid when the transfected or transduced nucleic acid imparts a phenotypic change in the cell and/or a change in an activity or function of the cell. The transforming nucleic acid can be integrated (covalently linked) into chromosomal DNA making up the genome of the cell or it can be present as a stable plasmid.

As used herein, the term “polypeptide” encompasses both peptides and proteins, unless indicated otherwise.

A “fusion protein” is a polypeptide produced when two heterologous nucleotide sequences or fragments thereof coding for two (or more) different polypeptides not found fused together in nature are fused together in the correct translational reading frame. Illustrative fusion polypeptides include fusions of a polypeptide of the invention (or a fragment thereof) to all or a portion of glutathione-S-transferase, maltose-binding protein, or a reporter protein (e.g., Green Fluorescent Protein, β-glucuronidase, β-galactosidase, luciferase, etc.), hemagglutinin, c-myc, FLAG epitope, etc.

As used herein, a “functional” polypeptide or “functional fragment” is one that substantially retains at least one biological activity normally associated with that polypeptide (e.g., inhibition of mineralization or binding to one or more members of the TGF-p superfamily). In particular embodiments, the “functional” polypeptide or “functional fragment” substantially retains all of the activities possessed by the unmodified peptide. By “substantially retains” biological activity, it is meant that the polypeptide retains at least about 50%, 60%, 75%, 85%, 90%, 95%, 97%, 98%, 99%, or more, of the biological activity of the native polypeptide (and can even have a higher level of activity than the native polypeptide). A “non-functional” polypeptide is one that exhibits little or essentially no detectable biological activity normally associated with the polypeptide (e.g., at most, only an insignificant amount, e.g., less than about 10% or even 5%). Biological activities such as protein binding and inhibition of mineralization can be measured using assays that are well known in the art and as described herein.

By the term “express” or “expression” of a polynucleotide coding sequence, it is meant that the sequence is transcribed, and optionally, translated. Typically, according to the present invention, expression of a coding sequence of the invention will result in production of the polypeptide of the invention. The entire expressed polypeptide or fragment can also function in intact cells without purification.

The term “about,” as used herein when referring to a measurable value such as an amount of polypeptide, dose, time, temperature, enzymatic activity or other biological activity and the like, is meant to encompass variations of ±20%, ±10%, ±5%, ±1%, ±0.5%, or even ±0.1% of the specified amount.

### II. G11 and F12 Polynucleotides, Polypeptides, Expression Vectors, and Host Cells

The inventors have discovered and characterized polypeptides, and polynucleotides encoding the polypeptides, which have the properties of being expressed in osteoblasts, binding to one or more members of the TGF-β superfamily, and inhibiting mineralization of cells in which they are expressed. Each of the polypeptides comprises a signal peptide sequence at the N-terminus and two cysteine-rich domains near the C-terminus. The location of the cysteines within the cysteine-rich domains places these polypeptides in the chordin family.

The G11 polypeptide was identified in mouse cells. The polynucleotide encoding G11 has the nucleotide sequence of SEQ ID NO:1 and the polypeptide has the amino acid sequence of SEQ ID NO:2. The sequences have GenBank accession number DQ421811.

The F12 polypeptide was first identified in mouse cells. Three alternatively spliced forms of the F12 polynucleotide produce three distinct F12 polypeptides. The polynucleotide encoding F12-1 has the nucleotide sequence of SEQ ID NO:3 and the polypeptide has the amino acid sequence of SEQ ID NO:4. The sequences have GenBank accession number EF552208. The polynucleotide encoding F12-2 has the nucleotide sequence of SEQ ID NO:5 and the polypeptide has the amino acid sequence of SEQ ID NO:6. The sequences have GenBank accession number EF552209. The polynucleotide encoding F12-3 has the nucleotide sequence of SEQ ID NO:7 and the polypeptide has the amino acid sequence of SEQ ID NO:8. The sequences have GenBank accession number EF552210.

Two human homologs of F12 have been identified. The polynucleotide encoding human F12-1 has the nucleotide sequence of SEQ ID NO:9 and the polypeptide has the amino acid sequence of SEQ ID NO:10. The sequences have GenBank accession number EU541473. The polynucleotide encoding human F12-2 has the nucleotide sequence of SEQ ID NO:11 and the polypeptide has the amino acid sequence of SEQ ID NO:12. The sequences have GenBank accession number EF552207. All GenBank accession number sequence submissions are incorporated herein by reference. In case of any inconsistency between the sequences in the disclosed sequence identifier numbers and the GenBank accession numbers, the GenBank accession number sequences will be considered the correct sequences.

In representative embodiments, the invention provides isolated polynucleotides encoding a G11 or F12 polypeptide (or a functional fragment thereof) as well as the isolated G11 or F12 polypeptides (or a functional fragment thereof). The G11 or F12 polynucleotides and polypeptides of the invention encompass sequences from any species of interest (e.g., mammalian (such as human, simian, mouse, rat, lagomorph, bovine, ovine, caprine, porcine, equine, feline, canine, etc.), insect, yeast, avian, plants, etc.) as well as allelic variations, isoforms, splice variants and the like. The G11 or F12 polynucleotides and polypeptides also include modifications that result in functional polypeptides.

Indicia of “functional” G11 or F12 polypeptides include those measures disclosed herein (e.g., in the working Examples) as well as other assays and techniques known in the art for determining protein:protein interaction, inhibition of mineralization, and other activities associated with the function of the G11 or F12 polypeptide.

Thus, as one aspect, the invention provides an isolated polynucleotide encoding G11 or F12. In exemplary embodiments, the isolated polynucleotide comprises, consists essentially of, or consists of the nucleotide sequences of SEQ ID NO:1, SEQ ID NO:3, SEQ ID NO:5, SEQ ID NO:7, SEQ ID NO:9, or SEQ ID NO:11. In another embodiment, the isolated polynucleotide comprises, consists essentially of, or consists of a nucleotide sequence that is at least 70% identical, e.g., at least 75%, 80%, 85%, 90%, 95%, 96%, 97%, 98%, or 99% identical to the nucleotide sequences of SEQ ID NO:1, SEQ ID NO:3, SEQ ID NO:5, SEQ ID NO:7, SEQ ID NO:9, or SEQ ID NO:11. In a further embodiment, the isolated polynucleotide comprises, consists essentially of, or consists of a nucleotide sequence that hybridizes under stringent conditions to a polynucleotide comprising, consisting essentially of, or consisting of the nucleotide sequences of SEQ ID NO:1, SEQ ID NO:3, SEQ ID NO:5, SEQ ID NO:7, SEQ ID NO:9, or SEQ ID NO:11.

In one embodiment of the invention, the isolated polynucleotide comprises, consists essentially of, or consists of a nucleotide sequence that encodes a polypeptide comprising, consisting essentially of, or consisting of an amino acid sequence of SEQ ID NO:2, SEQ ID NO:4, SEQ ID NO:6, SEQ ID NO:8, SEQ ID NO:10, or SEQ ID NO:12. In another embodiment, the isolated polynucleotide comprises, consists essentially of, or consists of a nucleotide sequence that encodes a polypeptide comprising, consisting essentially of, or consisting of an amino acid sequence that is at least 70% identical, e.g., at least 75%, 80%, 85%, 90%, 95%, 96%, 97%, 98%, or 99% identical to the amino acid sequence of SEQ ID NO:2, SEQ ID NO:4, SEQ ID NO:6, SEQ ID NO:8, SEQ ID NO:10, or SEQ ID NO:12. In a further embodiment of the invention, the isolated polynucleotide comprises, consists essentially of, or consists of a nucleotide sequence that encodes a polypeptide comprising, consisting essentially of, or consisting of an amino acid sequence of SEQ ID NO:2, SEQ ID NO:4, SEQ ID NO:6, SEQ ID NO:8, SEQ ID NO:10, or SEQ ID NO:12 and differs from the nucleotide sequence of SEQ ID NO:1, SEQ ID NO:3, SEQ ID NO:5, SEQ ID NO:7, SEQ ID NO:9, or SEQ ID NO:11 due to the degeneracy of the genetic code.

Isolated polynucleotides of this invention include RNA, DNA (including cDNAs) and chimeras thereof. The isolated polynucleotides can further comprise modified nucleotides or nucleotide analogs.

In other embodiments, the invention provides a polynucleotide that encodes a functional fragment of a G11 or F12 polypeptide (e.g., a fragment of SEQ ID NO:2, SEQ ID NO:4, SEQ ID NO:6, SEQ ID NO:8, SEQ ID NO:10, or SEQ ID NO:12 and other fragments disclosed herein). Such polynucleotides will typically comprise at least about 8, 10, 12, 15, 20, 25, 30, 35, 40, 45, 50, 75, 100, 150, 200, or more contiguous nucleotides of a nucleotide sequence encoding the indicated G11 or F12 polypeptide and encodes a functional fragment thereof.

As yet a further aspect, the invention provides an isolated G11 or F12 polypeptide. In exemplary embodiments, the polypeptide comprises, consists essentially of, or consists of the amino acid sequence of SEQ ID NO:2, SEQ ID NO:4, SEQ ID NO:6, SEQ ID NO:8, SEQ ID NO:10, or SEQ ID NO:12 or a functional fragment thereof. In another embodiment, the isolated polypeptide comprises, consists essentially of, or consists of an amino acid sequence that is at least 70% identical, e.g., at least 75%, 80%, 85%, 90%, 95%, 96%, 97%, 98%, or 99% identical to the amino acid sequence of SEQ ID NO:2, SEQ ID NO:4, SEQ ID NO:6, SEQ ID NO:8, SEQ ID NO:10, or SEQ ID NO:12 or a functional fragment thereof.

The G11 or F12 polypeptides of the invention also include functional portions or fragments of a G11 or F12 polypeptide (e.g., functional fragments of SEQ ID NO:2, SEQ ID NO:4, SEQ ID NO:6, SEQ ID NO:8, SEQ ID NO:10, or SEQ ID NO:12 and other polypeptide fragments disclosed herein). The length of the fragment is not critical as long as it substantially retains the biological activity of the polypeptide. Illustrative fragments comprise at least about 4, 6, 8, 10, 12, 15, 20, 25, 30, 35, 40, 45, 50, 75, 100, 150, 200, or more contiguous amino acids of a G11 or F12 polypeptide.

The present inventors have discovered that the G11 or F12 polypeptides comprise a N-terminal signal peptide and two C-terminal cysteine-rich domains. In particular embodiments, the invention provides a functional fragment of a G11 or F12 polypeptide comprising one or both of the cysteine-rich domains and functional fragments without the signal peptide (and polynucleotides encoding the same).

Likewise, those skilled in the art will appreciate that the present invention also encompasses fusion proteins (and polynucleotide sequences encoding the same) comprising the G11 or F12 polypeptides of the invention (or a functional fragment thereof). For example, it may be useful to express the G11 or F12 polypeptide (or functional fragment) as a fusion protein that can be recognized by a commercially available antibody (e.g., FLAG motifs) or as a fusion protein that can otherwise be more easily purified (e.g., by addition of a poly-His tail). Additionally, fusion proteins that enhance the stability of the G11 or F12 polypeptide may be produced, e.g., fusion proteins comprising maltose binding protein (MBP) or glutathione-S-transferase. As another alternative, the fusion protein can comprise a reporter molecule. In other embodiments, the fusion protein can comprise a polypeptide that provides a function or activity that is the same as or different from the activity of G11 or F12, e.g., a targeting, binding, or enzymatic activity or function.

Likewise, it will be understood that the G11 or F12 polypeptides specifically disclosed herein will typically tolerate substitutions in the amino acid sequence and substantially retain biological activity. To identify polypeptides of the invention other than those specifically disclosed herein, amino acid substitutions may be based on any characteristic known in the art, including the relative similarity or differences of the amino acid side-chain substituents, for example, their hydrophobicity, hydrophilicity, charge, size, and the like.

Amino acid substitutions other than those disclosed herein may be achieved by changing the codons of the DNA sequence (or RNA sequence), according to the following codon table:

TABLE 1

| | |
|---|---|
| Amino Acid | Codons |

| | | | |
|---|---|---|---|
| Alanine | Ala | A | GCA GCC GCG GCT |
| Cysteine | Cys | C | TGC TGT |
| Aspartic acid | Asp | D | GAC GAT |
| Glutamic acid | Glu | E | GAA GAG |
| Phenylalanine | Phe | F | TTC TTT |
| Glycine | Gly | G | GGA GGC GGG GGT |
| Histidine | His | H | CAC CAT |
| Isoleucine | Ile | I | ATA ATC ATT |
| Lysine | Lys | K | AAA AAG |
| Leucine | Leu | L | TTA TTG CTA CTC CTG CTT |
| Methionine | Met | M | ATG |
| Asparagine | Asn | N | AAC AAT |
| Proline | Pro | P | CCA CCC CCG CCT |
| Glutamine | Gln | Q | CAA GAG |
| Arginine | Arg | R | AGA AGG CGA CGC CGG CGT |
| Serine | Ser | S | AGC ACT TCA TCC TCG TCT |
| Threonine | Thr | T | ACA ACC ACG ACT |
| Valine | Val | V | GTA GTC GTG GTT |
| Tryptophan | Trp | W | TGG |
| Tyrosine | Tyr | Y | TAC TAT |

In identifying amino acid sequences encoding G11 or F12 polypeptides other than those specifically disclosed herein, the hydropathic index of amino acids may be considered. The importance of the hydropathic amino acid index in conferring interactive biologic function on a protein is generally understood in the art (see, Kyte and Doolittle, J. Mol. Biol. 157:105 (1982); incorporated herein by reference in its entirety). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like.

Each amino acid has been assigned a hydropathic index on the basis of its hydrophobicity and charge characteristics (Kyte and Doolittle, id.), these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

Accordingly, the hydropathic index of the amino acid (or amino acid sequence) may be considered when modifying the G11 or F12 polypeptides specifically disclosed herein.

It is also understood in the art that the substitution of amino acids can be made on the basis of hydrophilicity. U.S. Pat. No. 4,554,101 (incorporated herein by reference in its entirety) states that the greatest local average hydrophilicity of a protein, as governed by the hydrophilicity of its adjacent amino acids, correlates with a biological property of the protein.

As detailed in U.S. Pat. No. 4,554,101, the following hydrophilicity values have been assigned to amino acid residues: arginine (+3.0); lysine (±3.0); aspartate (+3.0±1); glutamate (+3.0±1); serine (+0.3); asparagine (+0.2); glutamine (+0.2); glycine (0); threonine (−0.4); proline (−0.5±I); alanine (−0.5); histidine (−0.5); cysteine (−1.0); methionine (−1.3); valine (−1.5); leucine (−1.8); isoleucine (−1.8); tyrosine (−2.3); phenylalanine (−2.5), tryptophan (−3.4).

Thus, the hydrophilicity of the amino acid (or amino acid sequence) may be considered when identifying additional G11 or F12 polypeptides beyond those specifically disclosed herein.

In embodiments of the invention, the polynucleotide encoding the G11 or F12 polypeptide (or functional fragment) will hybridize to the nucleic acid sequences specifically disclosed herein or fragments thereof (e.g., SEQ ID NO:1, SEQ ID NO:3, SEQ ID NO:5, SEQ ID NO:7, SEQ ID NO:9, or SEQ ID NO:11) under standard conditions as known by those skilled in the art and encode a functional G11 or F12 polypeptide or functional fragment thereof.

For example, hybridization of such sequences may be carried out under conditions of reduced stringency, medium stringency or even stringent conditions (e.g., conditions represented by a wash stringency of 35-40% formamide with 5× Denhardt's solution, 0.5% SDS and 1×SSPE at 37° C.; conditions represented by a wash stringency of 40-45% formamide with 5× Denhardt's solution, 0.5% SDS, and 1×SSPE at 42° C.; and conditions represented by a wash stringency of 50% formamide with 5× Denhardt's solution, 0.5% SDS and 1×SSPE at 42° C., respectively) to the polynucleotide sequences encoding the G11 or F12 polypeptides or functional fragments thereof specifically disclosed herein. See, e.g., Sambrook et al, Molecular Cloning: A Laboratory Manual 2nd Ed. (Cold Spring Harbor, N.Y., 1989).

In other embodiments, polynucleotide sequences encoding the G11 or F12 polypeptides of the invention have at least about 70%, 80%, 85%, 90%, 95%, 96%, 97%, 98%, 99% or higher sequence identity with the nucleic acid sequences specifically disclosed herein (or functional fragments thereof, as described above) and encode a functional G11 or F12 polypeptide or functional fragment thereof.

Further, it will be appreciated by those skilled in the art that there can be variability in the polynucleotides that encode the G11 or F12 polypeptides (and fragments thereof) of the present invention due to the degeneracy of the genetic code. The degeneracy of the genetic code, which allows different nucleic acid sequences to code for the same polypeptide, is well known in the literature (See, e.g., Table 1).

Likewise, the G11 or F12 polypeptides (and fragments thereof) of the invention include polypeptides that have at least about 70%, 80%, 85%, 90%, 95%, 96%, 97%, 98%, 99% or higher amino acid sequence identity with the polypeptide sequences specifically disclosed herein or fragments thereof (as described above).

As is known in the art, a number of different programs can be used to identify whether a polynucleotide or polypeptide has sequence identity or similarity to a known sequence. Sequence identity or similarity may be determined using standard techniques known in the art, including, but not limited to, the local sequence identity algorithm of Smith & Waterman, Adv. Appl. Math. 2:482 (1981), by the sequence identity alignment algorithm of Needleman & Wunsch, J. Mol. Biol. 48:443 (1970), by the search for similarity method of Pearson & Lipman, Proc. Natl. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Drive, Madison, Wis.), the Best Fit sequence program described by Devereux et al., Nucl. Acid Res. 12:387 (1984), preferably using the default settings, or by inspection.

An example of a useful algorithm is PILEUP. PILEUP creates a multiple sequence alignment from a group of related sequences using progressive, pairwise alignments. It can also plot a tree showing the clustering relationships used to create the alignment. PILEUP uses a simplification of the progressive alignment method of Feng & Doolittle, J. Mol. Evol. 35:351 (1987); the method is similar to that described by Higgins & Sharp, CABIOS 5:151 (1989).

Another example of a useful algorithm is the BLAST algorithm, described in Altschul et al., J. Mol. Biol. 215:403 (1990) and Karlin et al., Proc. Natl. Acad. Sci. USA 90:5873 (1993). A particularly useful BLAST program is the WU-BLAST-2 program which was obtained from Altschul et al., Meth. Enzymol., 266:460 (1996); blast.wust1/edu/blast/README.html. WU-BLAST-2 uses several search parameters, which are preferably set to the default values. The parameters are dynamic values and are established by the program itself depending upon the composition of the particular sequence and composition of the particular database against which the sequence of interest is being searched; however, the values may be adjusted to increase sensitivity.

An additional useful algorithm is gapped BLAST as reported by Altschul et al., Nucleic Acids Res. 25:3389 (1997).

A percentage amino acid sequence identity value is determined by the number of matching identical residues divided by the total number of residues of the “longer” sequence in the aligned region. The “longer” sequence is the one having the most actual residues in the aligned region (gaps introduced by WU-Blast-2 to maximize the alignment score are ignored).

In a similar manner, percent nucleic acid sequence identity with respect to the coding sequence of the polypeptides disclosed herein is defined as the percentage of nucleotide residues in the candidate sequence that are identical with the nucleotides in the polynucleotide specifically disclosed herein.

The alignment may include the introduction of gaps in the sequences to be aligned. In addition, for sequences which contain either more or fewer amino acids than the polypeptides specifically disclosed herein, it is understood that in one embodiment, the percentage of sequence identity will be determined based on the number of identical amino acids in relation to the total number of amino acids. Thus, for example, sequence identity of sequences shorter than a sequence specifically disclosed herein, will be determined using the number of amino acids in the shorter sequence, in one embodiment. In percent identity calculations relative weight is not assigned to various manifestations of sequence variation, such as insertions, deletions, substitutions, etc.

In one embodiment, only identities are scored positively (+1) and all forms of sequence variation including gaps are assigned a value of “0,” which obviates the need for a weighted scale or parameters as described below for sequence similarity calculations. Percent sequence identity can be calculated, for example, by dividing the number of matching identical residues by the total number of residues of the “shorter” sequence in the aligned region and multiplying by 100. The “longer” sequence is the one having the most actual residues in the aligned region.

Those skilled in the art will appreciate that the isolated polynucleotides encoding the G11 or F12 polypeptides of the invention will typically be associated with appropriate expression control sequences, e.g., transcription/translation control signals and polyadenylation signals.

It will further be appreciated that a variety of promoter/enhancer elements can be used depending on the level and tissue-specific expression desired. The promoter can be constitutive or inducible, depending on the pattern of expression desired. The promoter can be native or foreign and can be a natural or a synthetic sequence. By foreign, it is intended that the transcriptional initiation region is not found in the wild-type host into which the transcriptional initiation region is introduced. The promoter is chosen so that it will function in the target cell(s) of interest.

To illustrate, the G11 or F12 coding sequence can be operatively associated with a cytomegalovirus (CMV) major immediate-early promoter, an albumin promoter, an Elongation Factor 1-α (EF1-α) promoter, a PγK promoter, a MFG promoter, or a Rous sarcoma virus promoter.

Inducible promoter/enhancer elements include hormone-inducible and metal-inducible elements, and other promoters regulated by exogenously supplied compounds, including without limitation, the zinc-inducible metallothionein (MT) promoter; the dexamethasone (Dex)-inducible mouse mammary tumor virus (MMTV) promoter; the T7 polymerase promoter system (see WO 98/10088); the ecdysone insect promoter (No et al., Proc. Natl. Acad. Sci. USA 93:3346 (1996)); the tetracycline-repressible system (Gossen et al., Proc. Natl. Acad. Sci. USA 89:5547 (1992)); the tetracycline-inducible system (Gossen et al., Science 268:1766 (1995); see also Harvey et al., Curr. Opin. Chem. Biol. 2:512 (1998)); the RU486-inducible system (Wang et al., Nat. Biotech. 15:239 (1997); Wang et al., Gene Ther., 4:432 (1997)); and the rapamycin-inducible system (Magari et al., J. Clin. Invest. 100:2865 (1997)).

Other tissue-specific promoters or regulatory promoters include, but are not limited to, promoters that typically confer tissue-specificity in osteoblast lineage cells. These include, but are not limited to, promoters for osteocalcin, collagen, bone sialoprotein (BSP), Runx2, and osteoblast stimulating factor-1 (OSF-1).

Moreover, specific initiation signals are generally required for efficient translation of inserted protein coding sequences. These translational control sequences, which can include the ATG initiation codon and adjacent sequences, can be of a variety of origins, both natural and synthetic.

The present invention further provides cells comprising the isolated polynucleotides and polypeptides of the invention. The cell may be a cultured cell or a cell in vivo, e.g., for use in therapeutic methods, screening methods, methods for studying the biological action of the G11 or F12 polypeptides, in methods of producing G11 or F12 polypeptides, or in methods of maintaining or amplifying the polynucleotides of the invention, etc.

In particular embodiments, the cell is an untransformed cell or a cell from a bone-related cell line (e.g., osteogenic cells such as osteoblasts or precursors thereof, including pre-osteoblasts such as MC3T3-E1 cells). In other representative embodiments, the cell is a T cell, B cell, epithelial cell, endothelial cell, or muscle cell.

The isolated polynucleotide can be incorporated into an expression vector. Expression vectors compatible with various host cells are well known in the art and contain suitable elements for transcription and translation of nucleic acids. Typically, an expression vector contains an “expression cassette,” which includes, in the 5′ to 3′ direction, a promoter, a coding sequence encoding a G11 or F12 polypeptide or functional fragment thereof operatively associated with the promoter, and, optionally, a termination sequence including a stop signal for RNA polymerase and a polyadenylation signal for polyadenylase.

Non-limiting examples of promoters of this invention include CYC1, HIS3, GAL1, GAL4, GAL10, ADH1, PGK, PHO5, GAPDH, ADC1, TRP1, URA3, LEU2, ENO, TPI, and alkaline phosphatase promoters (useful for expression in Saccharomyces); AOX1 promoter (useful for expression in Pichia); β-lactamase, lac, ara, tet, trp, IP_{L}, IP_{R}, T7, tac, and trc promoters (useful for expression in Escherichia coli); light regulated-, seed specific-, pollen specific-, ovary specific-, pathogenesis or disease related-promoters, cauliflower mosaic virus 35S, CMV 35S minimal, cassaya vein mosaic virus (CsVMV), chlorophyll a/b binding protein, ribulose 1,5-bisphosphate carboxylase, shoot-specific promoters, root specific promoters, chitinase, stress inducible promoters, rice tungro bacilliform virus, plant super-promoter, potato leucine aminopeptidase, nitrate reductase, mannopine synthase, nopaline synthase, ubiquitin, zein protein, and anthocyanin promoters (useful for expression in plant cells).

Further examples of animal and mammalian promoters known in the art include, but are not limited to, the SV40 early (SV40e) promoter region, the promoter contained in the 3′ long terminal repeat (LTR) of Rous sarcoma virus (RSV), the promoters of the E1A or major late promoter (MLP) genes of adenoviruses (Ad), the cytomegalovirus (CMV) early promoter, the herpes simplex virus (HSV) thymidine kinase (TK) promoter, baculovirus IE1 promoter, elongation factor 1 alpha (EF1) promoter, phosphoglycerate kinase (PGK) promoter, ubiquitin (Ubc) promoter, an albumin promoter, the regulatory sequences of the mouse metallothionein-L promoter and transcriptional control regions, the ubiquitous promoters (HPRT, vimentin, α-actin, tubulin and the like), the promoters of the intermediate filaments (desmin, neurofilaments, keratin, GFAP, and the like), the promoters of therapeutic genes (of the MDR, CFTR or factor VIII type, and the like), pathogenesis and/or disease-related promoters, and promoters that exhibit tissue specificity, such as the elastase I gene control region, which is active in pancreatic acinar cells; the insulin gene control region active in pancreatic beta cells, the immunoglobulin gene control region active in lymphoid cells, the mouse mammary tumor virus control region active in testicular, breast, lymphoid and mast cells; the albumin gene promoter, the Apo AI and Apo AII control regions active in liver, the alpha-fetoprotein gene control region active in liver, the alpha 1-antitrypsin gene control region active in the liver, the beta-globin gene control region active in myeloid cells, the myelin basic protein gene control region active in oligodendrocyte cells in the brain, the myosin light chain-2 gene control region active in skeletal muscle, and the gonadotropic releasing hormone gene control region active in the hypothalamus, the pyruvate kinase promoter, the villin promoter, the promoter of the fatty acid binding intestinal protein, the promoter of smooth muscle cell α-actin, and the like. In addition, any of these expression sequences of this invention can be modified by addition of enhancer and/or regulatory sequences and the like.

Enhancers that may be used in embodiments of the invention include but are not limited to: an SV40 enhancer, a cytomegalovirus (CMV) enhancer, an elongation factor I (EF1) enhancer, yeast enhancers, viral gene enhancers, and the like.

Termination control regions, i.e., terminator or polyadenylation sequences, may be derived from various genes native to the preferred hosts. In some embodiments of the invention, the termination control region may comprise or be derived from a synthetic sequence, a synthetic polyadenylation signal, an SV40 late polyadenylation signal, an SV40 polyadenylation signal, a bovine growth hormone (BGH) polyadenylation signal, viral terminator sequences, or the like.

Expression vectors can be designed for expression of polypeptides in prokaryotic or eukaryotic cells. For example, polypeptides can be expressed in bacterial cells such as E. Coli, insect cells (e.g., the baculovirus expression system), yeast cells, plant cells or mammalian cells. Some suitable host cells are discussed further in Goeddel, Gene Expression Technology: Methods in Enzymology 185, Academic Press, San Diego, Calif. (1990). Examples of bacterial vectors include pQE70, pQE60, pQE-9 (Qiagen), pBS, pD10, phagescript, psiX174, pbluescript SK, pbsks, pNH8A, pNH16a, pNH18A, pNH46A (Stratagene); ptrc99a, pKK223-3, pKK233-3, pDR540, and pRIT5 (Pharmacia). Examples of vectors for expression in the yeast S. cerevisiae include pYepSec1 (Baldari et al., EMBO J. 6:229 (1987)), pMFa (Kurjan and Herskowitz, Cell 30:933 (1982)), pJRY88 (Schultz et al., Gene 54:113 (1987)), and pYES2 (Invitrogen Corporation, San Diego, Calif.). Baculovirus vectors available for expression of nucleic acids to produce proteins in cultured insect cells (e.g., Sf9 cells) include the pAc series (Smith et al., Mol. Cell. Biol. 3:2156 (1983)) and the pVL series (Lucklow and Summers Virology 170:31 (1989)).

Examples of mammalian expression vectors include pWLNEO, pSV2CAT, pOG44, pXT1, pSG (Stratagene) pSVK3, PBPV, pMSG, PSVL (Pharmacia), pCDM8 (Seed, Nature 329:840 (1987)) and pMT2PC (Kaufman et al., EMBO J. 6:187 (1987)). When used in mammalian cells, the expression vector's control functions are often provided by viral regulatory elements. For example, commonly used promoters are derived from polyoma, adenovirus 2, cytomegalovirus and Simian Virus 40.

Viral vectors have been used in a wide variety of gene delivery applications in cells, as well as living animal subjects. Viral vectors that can be used include, but are not limited to, retrovirus, lentivirus, adeno-associated virus, poxvirus, alphavirus, baculovirus, vaccinia virus, herpes virus, Epstein-Barr virus, adenovirus, geminivirus, and caulimovirus vectors. Non-viral vectors include plasmids, liposomes, electrically charged lipids (cytofectins), nucleic acid-protein complexes, and biopolymers. In addition to a nucleic acid of interest, a vector may also comprise one or more regulatory regions, and/or selectable markers useful in selecting, measuring, and monitoring nucleic acid transfer results (delivery to specific tissues, duration of expression, etc.).

In addition to the regulatory control sequences discussed above, the recombinant expression vector can contain additional nucleotide sequences. For example, the recombinant expression vector can encode a selectable marker gene to identify host cells that have incorporated the vector.

Vector DNA can be introduced into prokaryotic or eukaryotic cells via conventional transformation or transfection techniques. As used herein, the terms “transformation” and “transfection” refer to a variety of art-recognized techniques for introducing foreign nucleic acids (e.g., DNA and RNA) into a host cell, including calcium phosphate or calcium chloride co-precipitation, DEAE-dextran-mediated transfection, lipofection, electroporation, microinjection, DNA-loaded liposomes, lipofectamine-DNA complexes, cell sonication, gene bombardment using high velocity microprojectiles, and viral-mediated transfection. Suitable methods for transforming or transfecting host cells can be found in Sambrook et al., Molecular Cloning: A Laboratory Manual 2nd Ed. (Cold Spring Harbor, N.Y., 1989), and other laboratory manuals.

If stable integration is desired, often only a small fraction of cells (in particular, mammalian cells) integrate the foreign DNA into their genome. In order to identify and select integrants, a nucleic acid that encodes a selectable marker (e.g., resistance to antibiotics) can be introduced into the host cells along with the nucleic acid of interest. Preferred selectable markers include those that confer resistance to drugs, such as G418, hygromycin and methotrexate. Nucleic acids encoding a selectable marker can be introduced into a host cell on the same vector as that comprising the nucleic acid of interest or can be introduced on a separate vector. Cells stably transfected with the introduced nucleic acid can be identified by drug selection (e.g., cells that have incorporated the selectable marker gene will survive, while the other cells die).

The polynucleotide can also be introduced into a plant, plant cell or protoplast and, optionally, the isolated nucleic acid encoding the polypeptide is integrated into the nuclear or plastidic genome. Plant transformation is known as the art. See, in general, Meth. Enzymol. Vol. 153 (“Recombinant DNA Part D”) 1987, Wu and Grossman Eds., Academic Press and European Patent Application EP 693554.

### G11 and F12 Antibodies

As yet a further embodiment, the invention provides antibodies and antibody fragments that specifically bind to G11 or F12 polypeptides.

The term “antibody” or “antibodies” as used herein refers to all types of immunoglobulins, including IgG, IgM, IgA, IgD, and IgE. The antibody can be monoclonal or polyclonal and can be of any species of origin, including (for example) mouse, rat, rabbit, horse, goat, sheep, camel, or human, or can be a chimeric antibody. See, e.g., Walker et al, Molec. Immunol. 26:403 (1989). The antibodies can be recombinant monoclonal antibodies produced according to the methods disclosed in U.S. Pat. No. 4,474,893 or U.S. Pat. No. 4,816,567. The antibodies can also be chemically constructed according to the method disclosed in U.S. Pat. No. 4,676,980.

Antibody fragments included within the scope of the present invention include, for example, Fab, Fab′, F(ab′)₂, and Fv fragments; diabodies; linear antibodies; single-chain antibody molecules; and multispecific antibodies formed from antibody fragments. Such fragments can be produced by known techniques. For example, F(ab′)₂ fragments can be produced by pepsin digestion of the antibody molecule, and Fab fragments can be generated by reducing the disulfide bridges of the F(ab′)₂ fragments. Alternatively, Fab expression libraries can be constructed to allow rapid and easy identification of monoclonal Fab fragments with the desired specificity (Huse et al., Science 254:1275 (1989)).

Antibodies of the invention may be altered or mutated for compatibility with species other than the species in which the antibody was produced. For example, antibodies may be humanized or camelized. Humanized forms of non-human (e.g., murine) antibodies are chimeric immunoglobulins, immunoglobulin chains or fragments thereof (such as Fv, Fab, Fab′, F(ab′)₂ or other antigen-binding subsequences of antibodies) which contain minimal sequence derived from non-human immunoglobulin. Humanized antibodies include human immunoglobulins (recipient antibody) in which residues from a complementarity determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fe), typically that of a human immunoglobulin (Jones et al., Nature 321:522 (1986); Riechmann et al., Nature, 332:323 (1988); and Presta, Curr. Op. Struct. Biol. 2:593 (1992)).

Methods for humanizing non-human antibodies are well known in the art. Generally, a humanized antibody has one or more amino acid residues introduced into it from a source which is non-human. These non-human amino acid residues are often referred to as “import” residues, which are typically taken from an “import” variable domain. Humanization can be essentially performed following the method of Winter and co-workers (Jones et al., Nature 321:522 (1986); Riechmann et al., Nature 332:323 (1988); Verhoeyen et al., Science 239:1534 (1988)), by substituting rodent CDRs or CDR sequences for the corresponding sequences of a human antibody. Accordingly, such “humanized” antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

Human antibodies can also be produced using various techniques known in the art, including phage display libraries (Hoogenboom and Winter, J. Mol. Biol. 227:381 (1991); Marks et al., J. Mol. Biol. 222:581 (1991)). The techniques of Cole et al. and Boerner et al. are also available for the preparation of human monoclonal antibodies (Cole et al., Monoclonal Antibodies and Cancer Therapy, Alan R. Liss, p. 77 (1985) and Boerner et al., J. Immunol. 147:86 (1991)). Similarly, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10:779 (1992); Lonberg et al., Nature 368:856 (1994); Morrison, Nature 368:812 (1994); Fishwild et al., Nature Biotechnol. 14:845 (1996); Neuberger, Nature Biotechnol. 14:826 (1996); Lonberg and Huszar, Intern. Rev. Immunol. 13:65 (1995).

Polyclonal antibodies used to carry out the present invention can be produced by immunizing a suitable animal (e.g., rabbit, goat, etc.) with an antigen to which a monoclonal antibody to the target binds, collecting immune serum from the animal, and separating the polyclonal antibodies from the immune serum, in accordance with known procedures.

Monoclonal antibodies used to carry out the present invention can be produced in a hybridoma cell line according to the technique of Kohler and Milstein, Nature 265:495 (1975). For example, a solution containing the appropriate antigen can be injected into a mouse and, after a sufficient time, the mouse sacrificed and spleen cells obtained. The spleen cells are then immortalized by fusing them with myeloma cells or with lymphoma cells, typically in the presence of polyethylene glycol, to produce hybridoma cells. The hybridoma cells are then grown in a suitable medium and the supernatant screened for monoclonal antibodies having the desired specificity. Monoclonal Fab fragments can be produced in E. coli by recombinant techniques known to those skilled in the art. See, e.g., Huse, Science 246:1275 (1989).

Antibodies specific to the target polypeptide can also be obtained by phage display techniques known in the art.

Various immunoassays can be used for screening to identify antibodies having the desired specificity for the polypeptides of this invention. Numerous protocols for competitive binding or immunoradiometric assays using either polyclonal or monoclonal antibodies with established specificity are well known in the art. Such immunoassays typically involve the measurement of complex formation between an antigen and its specific antibody (e.g., antigen/antibody complex formation). A two-site, monoclonal-based immunoassay utilizing monoclonal antibodies reactive to two non-interfering epitopes on the proteins or peptides of this invention can be used as well as a competitive binding assay.

Antibodies can be conjugated to a solid support (e.g., beads, plates, slides or wells formed from materials such as latex or polystyrene) in accordance with known techniques. Antibodies can likewise be conjugated to detectable groups such as radiolabels (e.g., ³⁵S, ¹²⁵I, ¹³¹I), enzyme labels (e.g., horseradish peroxidase, alkaline phosphatase), and fluorescence labels (e.g. fluorescein) in accordance with known techniques. Determination of the formation of an antibody/antigen complex in the methods of this invention can be by detection of, for example, precipitation, agglutination, flocculation, radioactivity, color development or change, fluorescence, luminescence, etc., as is well known in the art.

### III. Applications of the Present Invention

G11 and F12 polynucleotides, polypeptides, antibodies, cells and other reagents have a wide variety of uses, both in vitro and in vivo. For example, in representative embodiments, these reagents may be used in vitro or in vivo (e.g., in an animal model) to study the processes of mineralization, bone formation, and bone loss. Further, “knock in” and “knock out” animals can be used as animal models of disease or as screening tools (discussed more below) for compounds that interact with the G11 or F12 genes or polypeptides.

In one embodiment, the invention relates to methods of inhibiting mineralization in a cell by delivering to the cell G11 or F12 polynucleotides or polypeptides. In another embodiment of the invention, the invention relates to methods of promoting mineralization in a cell by inhibiting the activity of G11 or F12 polypeptides. The cells may be isolated cells or cells within an animal, e.g., a laboratory animal.

The invention can also be used to achieve therapeutic effects. The G11 and F12 polynucleotides and polypeptides are implicated in the regulation of diseases and conditions that relate to mineralization, bone formation and bone loss. Agents that increase G11 or F12 activity (including G11 or F12 polypeptides or polynucleotides) are useful for the prevention and/or treatment of diseases or conditions associated with excess bone formation or increased bone mass or where bone formation is undesirable, for example, scoliosis, disproportionate long bone growth, bone growth disturbance due to radiotherapy, prevention of bone growth in fractures before they are set or during the interval between removal and replacement of a tissue implant (e.g., artificial joint), or prevention of mineralization of tissue transplants.

Inhibitors of G11 or F12 activity (e.g., antibodies, antisense polynucleotides, siRNA) are useful for the prevention and/or treatment of diseases associated with loss in bone mass or conditions in which an increase in bone mass is desired, for example, primary osteoporosis, secondary osteoporosis, bone metastasis of cancer, hypercalcemia, Paget's disease, bone loss, osteonecrosis, osteoarthritis, rheumatoid arthritis, osteomalacia, achondroplasia, osteochondritis, hyperparathyroidism, osteogenesis imperfecta, congenital hypophosphatasia, fibromatous lesions, fibrous displasia, multiple myeloma, abnormal bone turnover, osteolytic bone disease, periodontal disease, bone fractures, and bone defects. They can also play a role in clinically-induced conditions such as surgery and transplantation, e.g., formation after bone operation or as alternative treatment for bone grafting.

According to the present invention, the activity of one or more G11 or F12 polypeptide can be modulated (e.g., increased or decreased) to treat the above-mentioned conditions. The activity of G11 or F12 polypeptides can be directly regulated at the nucleic acid (DNA or RNA) or protein level. Alternatively, or additionally, the activity of G11 or F12 polypeptides can be indirectly modulated by regulating factors that are upstream or downstream in pathways involved in G11 or F12 activity or by regulating any other factor which results in modulation of G11 or F12 activity. Further, interaction domains of G11 or F12 polypeptides with other polypeptides can be used to alter the function of either G11 or F12 or their interaction partners. Interaction sites can be defined and used to identify small molecules that can mimic this interaction or block this interaction.

According to the foregoing methods, one or more G11 or F12 polypeptides (or functional fragment thereof) can be introduced into a cell or administered to a subject. Alternatively, a polynucleotide encoding the polypeptide(s) (or functional fragment(s)) can be delivered so that the polypeptide(s) is produced in the cell or subject. As described in more detail hereinbelow, these polypeptides (or fragments thereof) can be used to screen for small molecules that can interact with them to enhance or block their function.

It will be apparent to those skilled in the art that any suitable vector can be used to deliver the polynucleotide to a cell or subject. The choice of delivery vector can be made based on a number of factors known in the art, including age and species of the target host, in vitro versus in vivo delivery, level and persistence of expression desired, intended purpose (e.g., for therapy or screening), the target cell or organ, route of delivery, size of the isolated polynucleotide, safety concerns, and the like.

Suitable vectors include plasmid vectors, viral vectors (e.g., retrovirus, alphavirus; vaccinia virus; adenovirus, adeno-associated virus and other parvoviruses, lentivirus, poxvirus, or herpes simplex virus), lipid vectors, poly-lysine vectors, synthetic polyamino polymer vectors, and the like.

Any viral vector that is known in the art can be used in the present invention. Protocols for producing recombinant viral vectors and for using viral vectors for nucleic acid delivery can be found in Ausubel et al., Current Protocols in Molecular Biology (Green Publishing Associates, Inc. and John Wiley & Sons, Inc., New York) and other standard laboratory manuals (e.g., Vectors for Gene Therapy. In: Current Protocols in Human Genetics. John Wiley and Sons, Inc.: 1997).

Non-viral transfer methods can also be employed. Many non-viral methods of nucleic acid transfer rely on normal mechanisms used by mammalian cells for the uptake and intracellular transport of macromolecules. In particular embodiments, non-viral nucleic acid delivery systems rely on endocytic pathways for the uptake of the nucleic acid molecule by the targeted cell. Exemplary nucleic acid delivery systems of this type include liposomal derived systems, poly-lysine conjugates, and artificial viral envelopes.

In particular embodiments, plasmid vectors are used in the practice of the present invention. For example, naked plasmids can be introduced into muscle cells by injection into the tissue. Expression can extend over many months, although the number of positive cells is typically low (Wolff et al., Science 247:247 (1989)). Cationic lipids have been demonstrated to aid in introduction of nucleic acids into some cells in culture (Felgner and Ringold, Nature 337:387 (1989)). Injection of cationic lipid plasmid DNA complexes into the circulation of mice has been shown to result in expression of the DNA in lung (Brigham et al., Am. J. Med. Sci. 298:278 (1989)). One advantage of plasmid DNA is that it can be introduced into non-replicating cells.

In a representative embodiment, a nucleic acid molecule (e.g., a plasmid) can be entrapped in a lipid particle bearing positive charges on its surface and, optionally, tagged with antibodies against cell surface antigens of the target tissue (Mizuno et al., No Shinkei Geka 20:547 (1992); PCT publication WO 91/06309; Japanese patent application 1047381; and European patent publication EP-A-43075).

Liposomes that consist of amphiphilic cationic molecules are useful as non-viral vectors for nucleic acid delivery in vitro and in vivo (reviewed in Crystal, Science 270:404 (1995); Blaese et al., Cancer Gene Ther. 2:291 (1995); Behr et al., Bioconjugate Chem. 5:382 (1994); Remy et al., Bioconjugate Chem. 5:647 (1994); and Gao et al., Gene Therapy 2:710 (1995)). The positively charged liposomes are believed to complex with negatively charged nucleic acids via electrostatic interactions to form lipid:nucleic acid complexes. The lipid:nucleic acid complexes have several advantages as nucleic acid transfer vectors. Unlike viral vectors, the lipid:nucleic acid complexes can be used to transfer expression cassettes of essentially unlimited size. Since the complexes lack proteins, they can evoke fewer immunogenic and inflammatory responses. Moreover, they cannot replicate or recombine to form an infectious agent and have low integration frequency. A number of publications have demonstrated that amphiphilic cationic lipids can mediate nucleic acid delivery in vivo and in vitro (Felgner et al., Proc. Natl. Acad. Sci. USA 84:7413 (1987); Loeffler et al., Meth. Enzymol. 217:599 (1993); Felgner et al., J. Biol. Chem. 269:2550 (1994)).

Several groups have reported the use of amphiphilic cationic lipid:nucleic acid complexes for in vivo transfection both in animals and in humans (reviewed in Gao et al., Gene Therapy 2:710 (1995); Zhu et al., Science 261:209 (1993); and Thierry et al., Proc. Natl. Acad. Sci. USA 92:9742 (1995)). U.S. Pat. No. 6,410,049 describes a method of preparing cationic lipid:nucleic acid complexes that have a prolonged shelf life.

In other embodiments, it is desired to reduce or inhibit the activity of one or more G11 or F12 polypeptides. The activity of G11 or F12 polypeptides can be inhibited at the nucleic acid or protein level. Alternatively, or additionally, the activity of G11 or F12 polypeptides can be indirectly inhibited by regulating factors that are upstream or downstream in pathways involved in G11 or F12 activity or by regulating any other factor which results in inhibition of G11 or F12 activity.

Numerous methods for reducing the activity of one or more G11 or F12 polypeptides in vitro or in vivo are known. For example, the coding and noncoding nucleotide sequences for G11 or F12 polynucleotides are disclosed herein. An antisense nucleotide sequence or nucleic acid encoding an antisense nucleotide sequence can be generated to any portion thereof in accordance with known techniques.

The term “antisense nucleotide sequence” or “antisense oligonucleotide” as used herein, refers to a nucleotide sequence that is complementary to a specified DNA or RNA sequence. Antisense oligonucleotides and nucleic acids that express the same can be made in accordance with conventional techniques. See, e.g., U.S. Pat. No. 5,023,243 to Tullis; U.S. Pat. No. 5,149,797 to Pederson et al.

As illustrative examples of an antisense nucleotide sequence that can be used to carry out the invention is a nucleotide sequence that is complementary to the nucleotide sequences of SEQ ID NO:1, SEQ ID NO:3, SEQ ID NO:5, SEQ ID NO:7, SEQ ID NO:9, or SEQ ID NO:11 (or a portion thereof of at least 10, 20, 40, 50, 75, 100, 150, 200, 300, or 500 contiguous bases) and will reduce the level of polypeptide production.

Those skilled in the art will appreciate that it is not necessary that the antisense nucleotide sequence be fully complementary to the target sequence as long as the degree of sequence similarity is sufficient for the antisense nucleotide sequence to hybridize to its target and reduce production of the polypeptide. As is known in the art, a higher degree of sequence similarity is generally required for short antisense nucleotide sequences, whereas a greater degree of mismatched bases will be tolerated by longer antisense nucleotide sequences.

In representative embodiments of the invention, the antisense nucleotide sequence will hybridize to the nucleotide sequences encoding the G11 or F12 polypeptides specifically disclosed herein (e.g. SEQ ID NO:2, SEQ ID NO:4, SEQ ID NO:6, SEQ ID NO:8, SEQ ID NO:10, or SEQ ID NO:12 or portions thereof) and will reduce the level of polypeptide production.

For example, hybridization of such nucleotide sequences can be carried out under conditions of reduced stringency, medium stringency or even stringent conditions (e.g., conditions represented by a wash stringency of 35-40% formamide with 5× Denhardt's solution, 0.5% SDS and 1×SSPE at 37° C.; conditions represented by a wash stringency of 40-45% formamide with 5× Denhardt's solution, 0.5% SDS, and 1×SSPE at 42° C.; and/or conditions represented by a wash stringency of 50% formamide with 5× Denhardt's solution, 0.5% SDS and 1×SSPE at 42° C., respectively) to the nucleotide sequences specifically disclosed herein. See, e.g., Sambrook et al., Molecular Cloning: A Laboratory Manual 2nd Ed. (Cold Spring Harbor, N.Y., 1989).

In other embodiments, antisense nucleotide sequences of the invention have at least about 70%, 80%, 90%, 95%, 97%, 98% or higher sequence similarity with the complement of the coding sequences specifically disclosed herein and will reduce the level of polypeptide production.

In other embodiments, the antisense nucleotide sequence can be directed against any coding sequence, the silencing of which results in a modulation of a G11 or F12 polypeptide.

The length of the antisense nucleotide sequence (i.e., the number of nucleotides therein) is not critical as long as it binds selectively to the intended location and reduces transcription and/or translation of the target sequence, and can be determined in accordance with routine procedures. In general, the antisense nucleotide sequence will be from about eight, ten or twelve nucleotides in length up to about 20, 30, 50, 75 or 100 nucleotides, or longer, in length.

An antisense nucleotide sequence can be constructed using chemical synthesis and enzymatic ligation reactions by procedures known in the art. For example, an antisense nucleotide sequence can be chemically synthesized using naturally occurring nucleotides or various modified nucleotides designed to increase the biological stability of the molecules or to increase the physical stability of the duplex formed between the antisense and sense nucleotide sequences, e.g., phosphorothioate derivatives and acridine substituted nucleotides can be used. Examples of modified nucleotides which can be used to generate the antisense nucleotide sequence include 5-fluorouracil, 5-bromouracil, 5-chlorouracil, 5-iodouracil, hypoxanthine, xanthine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleotide sequence can be produced using an expression vector into which a nucleic acid has been cloned in an antisense orientation (i.e., RNA transcribed from the inserted nucleic acid will be of an antisense orientation to a target nucleic acid of interest).

The antisense nucleotide sequences of the invention further include nucleotide sequences wherein at least one, or all, of the internucleotide bridging phosphate residues are modified phosphates, such as methyl phosphonates, methyl phosphonothioates, phosphoromorpholidates, phosphoropiperazidates and phosphoramidates. For example, every other one of the internucleotide bridging phosphate residues can be modified as described. In another non-limiting example, the antisense nucleotide sequence is a nucleotide sequence in which one, or all, of the nucleotides contain a 2′ lower alkyl moiety (e.g., C₁-C₄, linear or branched, saturated or unsaturated alkyl, such as methyl, ethyl, ethenyl, propyl, 1-propenyl, 2-propenyl, and isopropyl). For example, every other one of the nucleotides can be modified as described. See also, Furdon et al., Nucleic Acids Res. 17:9193 (1989); Agrawal et al., Proc. Natl. Acad. Sci. USA 87:1401 (1990); Baker et al., Nucleic Acids Res. 18:3537 (1990); Sproat et al, Nucleic Acids Res. 17:3373 (1989); Walder and Walder, Proc. Natl. Acad. Sci. USA 85:5011 (1988); incorporated by reference herein in their entireties for their teaching of methods of making antisense molecules, including those containing modified nucleotide bases).

Triple helix base-pairing methods can also be employed to inhibit production of G11 or F12 polypeptides. Triple helix pairing is believed to work by inhibiting the ability of the double helix to open sufficiently for the binding of polymerases, transcription factors, or regulatory molecules. Recent therapeutic advances using triplex DNA have been described in the literature (e.g., Gee et al., (1994) In: Huber et al., Molecular and Immunologic Approaches, Futura Publishing Co., Mt. Kisco, N.Y.).

Small Interference (si) RNA, also known as RNA interference (RNAi) molecules, provides another approach for modulating G11 or F12 polypeptide activity. The siRNA can be directed against the G11 or F12 polynucleotide sequence or any other sequence that results in modulation of G11 or F12 activity.

siRNA is a mechanism of post-transcriptional gene silencing in which double-stranded RNA (dsRNA) corresponding to a coding sequence of interest is introduced into a cell or an organism, resulting in degradation of the corresponding mRNA, The mechanism by which siRNA achieves gene silencing has been reviewed in Sharp et al., Genes Dev. 15:485 (2001); and Hammond et al., Nature Rev. Gen. 2:110 (2001)). The siRNA effect persists for multiple cell divisions before gene expression is regained. siRNA is therefore a powerful method for making targeted knockouts or “knockdowns” at the RNA level. siRNA has proven successful in human cells, including human embryonic kidney and HeLa cells (see, e.g., Elbashir et al., Nature 411:494 (2001)). In one embodiment, silencing can be induced in mammalian cells by enforcing endogenous expression of RNA hairpins (see Paddison et al., Proc. Natl. Acad. Sci. USA 99:1443 (2002)). In another embodiment, transfection of small (21-23 nt) dsRNA specifically inhibits nucleic acid expression (reviewed in Caplen, Trends Biotechnol. 20:49 (2002)).

siRNA technology utilizes standard molecular biology methods. dsRNA corresponding to all or a part of a target coding sequence to be inactivated can be produced by standard methods, e.g., by simultaneous transcription of both strands of a template DNA (corresponding to the target sequence) with T7 RNA polymerase. Kits for production of dsRNA for use in siRNA are available commercially, e.g., from New England Biolabs, Inc. Methods of transfection of dsRNA or plasmids engineered to make dsRNA are routine in the art.

In particular embodiments, the siRNA molecules comprise SEQ ID NO:1, SEQ ID NO:3, SEQ ID NO:5; SEQ ID NO:7, SEQ ID NO:9, and/or SEQ ID NO:11 or fragments thereof.

Silencing effects similar to those produced by siRNA have been reported in mammalian cells with transfection of a mRNA-cDNA hybrid construct (Lin et al., Biochem. Biophys. Res. Commun. 281:639 (2001)), providing yet another strategy for silencing a coding sequence of interest.

G11 or F12 polypeptide activity can also be inhibited using ribozymes. Ribozymes are RNA-protein complexes that cleave nucleic acids in a site-specific fashion. Ribozymes have specific catalytic domains that possess endonuclease activity (Kim et al., Proc. Natl. Acad. Sci. USA 84:8788 (1987); Gerlach et al., Nature 328:802 (1987); Forster and Symons, Cell 49:211 (1987)). For example, a large number of ribozymes accelerate phosphoester transfer reactions with a high degree of specificity, often cleaving only one of several phosphoesters in an oligonucleotide substrate (Michel and Westhof, J. Mol. Biol. 216:585 (1990); Reinhold-Hurek and Shub, Nature 357:173 (1992)). This specificity has been attributed to the requirement that the substrate bind via specific base-pairing interactions to the internal guide sequence (“IGS”) of the ribozyme prior to chemical reaction.

Ribozyme catalysis has primarily been observed as part of sequence-specific cleavage/ligation reactions involving nucleic acids (Joyce, Nature 338:217 (1989)). For example, U.S. Pat. No. 5,354,855 reports that certain ribozymes can act as endonucleases with a sequence specificity greater than that of known ribonucleases and approaching that of the DNA restriction enzymes. Thus, sequence-specific ribozyme-mediated inhibition of gene expression may be particularly suited to therapeutic applications (Scanlon et al., Proc. Natl. Acad. Sci. USA 88:10591 (1991); Sarver et al., Science 247:1222 (1990); Sioud et al., J. Mol. Biol. 223:831 (1992)).

G11 or F12 polypeptide activity can further be modulated by interaction with an antibody or antibody fragment. The antibody or antibody fragment can bind to the G11 or F12 polypeptide or to any other polypeptide of interest, as long as the binding between the antibody or the antibody fragment and the target polypeptide results in modulation of the G11 or F12 polypeptide activity. Antibodies and antibody fragments are as described in more detail hereinabove.

Furthermore, the present invention provides a method of modulating the activity of a G11 or F12 polypeptide (e.g., for therapy or other purposes described above), comprising administering to a cell or to a subject a compound that modulates the activity of a G11 or F12 polypeptide, the compound administered in an amount effective to modulate the activity of the G11 or F12 polypeptide. The compound can enhance or inhibit the activity of the G11 or F12 polypeptide. Further, the compound can interact directly with the G11 or F12 polypeptide or at the nucleic acid (DNA or RNA) level to modulate the activity of the polypeptide. Alternatively, the compound can interact with any other polypeptide, nucleic acid or other molecule if such interaction results in a modulation of the activity of the G11 or F12 polypeptide.

The term “compound” as used herein is intended to be interpreted broadly and encompasses organic and inorganic molecules. Organic compounds include, but are not limited to, polypeptides, lipids, carbohydrates, coenzymes, and nucleic acid molecules (e.g., gene delivery vectors, antisense oligonucleotides, siRNA, all as described above).

Polypeptides include, but are not limited to, antibodies (described in more detail above) and enzymes. Nucleic acids include, but are not limited to, DNA, RNA and DNA-RNA chimeric molecules. Suitable RNA molecules include siRNA, antisense RNA molecules and ribozymes (all of which are described in more detail above). The nucleic acid can further encode any polypeptide such that administration of the nucleic acid and production of the polypeptide results in a modulation of the activity of a G11 or F12 polypeptide.

The compound can further be a compound that is identified by any of the screening methods described below.

The compounds of the present invention can optionally be administered in conjunction with other therapeutic agents. The additional therapeutic agents can be administered concurrently with the compounds of the invention. As used herein, the word “concurrently” means sufficiently close in time to produce a combined effect (that is, concurrently can be simultaneously, or it can be two or more events occurring within a short time period before or after each other). In one embodiment, the compounds of the invention are administered in conjunction with promoters of bone formation or inhibitors of bone loss, such as bisphosphonates, estrogen agonists/antagonists, phosphodiesterase-4 inhibitors, vitamin D analogs, calcium, fluoride, calcitonin, isoflavones, anabolic steroids, vitamin K, cathepsin K, prostaglandins, statins, parathyroid hormones, bone morphogenetic proteins, TGF-β and TGF-β family members, fibroblast growth factors, interleukin-1 inhibitors, and TNFα inhibitors.

As a further aspect, the invention provides pharmaceutical formulations and methods of administering the same to achieve any of the therapeutic effects (e.g., inhibition of bone formation, inhibition of bone loss, etc.) discussed above. The pharmaceutical formulation may comprise any of the reagents discussed above in a pharmaceutically acceptable carrier, e.g., a polynucleotide encoding a G11 or F12 polypeptide or a fragment thereof, a G11 or F12 polypeptide or fragment thereof, an antibody against a G11 or F12 polypeptide, an antisense oligonucleotide, an siRNA molecule, a ribozyme, or any other compound that modulates the activity of a G11 or F12 polypeptide including compounds identified by the screening methods described herein. Small molecules or peptidomimetics that can bind to certain domains of a G11 or F12 polypeptide to enhance or block the function of the polypeptide is another pharmaceutical approach.

By “pharmaceutically acceptable” it is meant a material that is not biologically or otherwise undesirable, i.e., the material can be administered to a subject without causing any undesirable biological effects such as toxicity.

The formulations of the invention can optionally comprise medicinal agents, pharmaceutical agents, carriers, adjuvants, dispersing agents, diluents, and the like.

The compounds of the invention can be formulated for administration in a pharmaceutical carrier in accordance with known techniques. See, e.g., Remington, The Science And Practice of Pharmacy (9^{th} Ed. 1995). In the manufacture of a pharmaceutical formulation according to the invention, the compound (including the physiologically acceptable salts thereof) is typically admixed with, inter alia, an acceptable carrier. The carrier can be a solid or a liquid, or both, and is preferably formulated with the compound as a unit-dose formulation, for example, a tablet, which can contain from 0.01 or 0.5% to 95% or 99% by weight of the compound. One or more compounds can be incorporated in the formulations of the invention, which can be prepared by any of the well-known techniques of pharmacy.

A further aspect of the invention is a method of treating subjects in vivo, comprising administering to a subject a pharmaceutical composition comprising a compound of the invention in a pharmaceutically acceptable carrier, wherein the pharmaceutical composition is administered in a therapeutically effective amount. Administration of the compounds of the present invention to a human subject or an animal in need thereof can be by any means known in the art for administering compounds.

The formulations of the invention include those suitable for oral, rectal, topical, buccal (e.g., sub-lingual), vaginal, parenteral (e.g., subcutaneous, intramuscular including skeletal muscle, cardiac muscle, diaphragm muscle and smooth muscle, intradermal, intravenous, intraperitoneal), topical (i.e., both skin and mucosal surfaces, including airway surfaces), intranasal, transdermal, intraarticular, intrathecal, and inhalation administration, administration to the liver by intraportal delivery, as well as direct organ injection (e.g., into the liver, into the brain for delivery to the central nervous system, into the pancreas). The most suitable route in any given case will depend on the nature and severity of the condition being treated and on the nature of the particular compound which is being used.

For injection, the carrier will typically be a liquid, such as sterile pyrogen-free water, pyrogen-free phosphate-buffered saline solution, bacteriostatic water, or Cremophor EL[R] (BASF, Parsippany, N.J.). For other methods of administration, the carrier can be either solid or liquid.

For oral administration, the compound can be administered in solid dosage forms, such as capsules, tablets, and powders, or in liquid dosage forms, such as elixirs, syrups, and suspensions. Compounds can be encapsulated in gelatin capsules together with inactive ingredients and powdered carriers, such as glucose, lactose, sucrose, mannitol, starch, cellulose or cellulose derivatives, magnesium stearate, stearic acid, sodium saccharin, talcum, magnesium carbonate and the like. Examples of additional inactive ingredients that can be added to provide desirable color, taste, stability, buffering capacity, dispersion or other known desirable features are red iron oxide, silica gel, sodium lauryl sulfate, titanium dioxide, edible white ink and the like. Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric-coated for selective disintegration in the gastrointestinal tract. Liquid dosage forms for oral administration can contain coloring and flavoring to increase patient acceptance.

Formulations suitable for buccal (sub-lingual) administration include lozenges comprising the compound in a flavored base, usually sucrose and acacia or tragacanth; and pastilles comprising the compound in an inert base such as gelatin and glycerin or sucrose and acacia.

Formulations of the present invention suitable for parenteral administration comprise sterile aqueous and non-aqueous injection solutions of the compound, which preparations are preferably isotonic with the blood of the intended recipient. These preparations can contain anti-oxidants, buffers, bacteriostats and solutes which render the formulation isotonic with the blood of the intended recipient. Aqueous and non-aqueous sterile suspensions can include suspending agents and thickening agents. The formulations can be presented in unit\dose or multi-dose containers, for example sealed ampoules and vials, and can be stored in a freeze-dried (lyophilized) condition requiring only the addition of the sterile liquid carrier, for example, saline or water-for-injection immediately prior to use.

Extemporaneous injection solutions and suspensions can be prepared from sterile powders, granules and tablets of the kind previously described. For example, in one aspect of the present invention, there is provided an injectable, stable, sterile composition comprising a compound of the invention, in a unit dosage form in a sealed container. The compound or salt is provided in the form of a lyophilizate which is capable of being reconstituted with a suitable pharmaceutically acceptable carrier to form a liquid composition suitable for injection thereof into a subject. The unit dosage form typically comprises from about 10 mg to about 10 grams of the compound or salt. When the compound or salt is substantially water-insoluble, a sufficient amount of emulsifying agent which is pharmaceutically acceptable can be employed in sufficient quantity to emulsify the compound or salt in an aqueous carrier. One such useful emulsifying agent is phosphatidyl choline.

Formulations suitable for rectal administration are preferably presented as unit dose suppositories. These can be prepared by admixing the compound with one or more conventional solid carriers, for example, cocoa butter, and then shaping the resulting mixture.

Formulations suitable for topical application to the skin preferably take the form of an ointment, cream, lotion, paste, gel, spray, aerosol, or oil. Carriers which can be used include petroleum jelly, lanoline, polyethylene glycols, alcohols, transdermal enhancers, and combinations of two or more thereof.

Formulations suitable for transdermal administration can be presented as discrete patches adapted to remain in intimate contact with the epidermis of the recipient for a prolonged period of time. Formulations suitable for transdernal administration can also be delivered by iontophoresis (see, for example, Tyle, Pharm. Res. 3:318 (1986)) and typically take the form of an optionally buffered aqueous solution of the compound. Suitable formulations comprise citrate or bis\tris buffer (pH 6) or ethanol/water and contain from 0.1 to 0.2M of the compound.

The compound can alternatively be formulated for nasal administration or otherwise administered to the lungs of a subject by any suitable means, e.g., administered by an aerosol suspension of respirable particles comprising the compound, which the subject inhales. The respirable particles can be liquid or solid. The term “aerosol” includes any gas-borne suspended phase, which is capable of being inhaled into the bronchioles or nasal passages. Specifically, aerosol includes a gas-borne suspension of droplets, as can be produced in a metered dose inhaler or nebulizer, or in a mist sprayer. Aerosol also includes a dry powder composition suspended in air or other carrier gas, which can be delivered by insufflation from an inhaler device, for example. See Ganderton & Jones, Drug Delivery to the Respiratory Tract, Ellis Horwood (1987); Gonda (1990) Critical Reviews in Therapeutic Drug Carrier Systems 6:273-313; and Raeburn et al., J. Pharmacol. Toxicol. Meth. 27:143 (1992). Aerosols of liquid particles comprising the compound can be produced by any suitable means, such as with a pressure-driven aerosol nebulizer or an ultrasonic nebulizer, as is known to those of skill in the art. See, e.g., U.S. Pat. No. 4,501,729. Aerosols of solid particles comprising the compound can likewise be produced with any solid particulate medicament aerosol generator, by techniques known in the pharmaceutical art.

Alternatively, one can administer the compound in a local rather than systemic manner, for example, in a depot or sustained-release formulation.

Further, the present invention provides liposomal formulations of the compounds disclosed herein and salts thereof. The technology for forming liposomal suspensions is well known in the art. When the compound or salt thereof is an aqueous-soluble salt, using conventional liposome technology, the same can be incorporated into lipid vesicles. In such an instance, due to the water solubility of the compound or salt, the compound or salt will be substantially entrained within the hydrophilic center or core of the liposomes. The lipid layer employed can be of any conventional composition and can either contain cholesterol or can be cholesterol-free. When the compound or salt of interest is water-insoluble, again employing conventional liposome formation technology, the salt can be substantially entrained within the hydrophobic lipid bilayer which forms the structure of the liposome. In either instance, the liposomes which are produced can be reduced in size, as through the use of standard sonication and homogenization techniques.

The liposomal formulations containing the compounds disclosed herein or salts thereof, can be lyophilized to produce a lyophilizate which can be reconstituted with a pharmaceutically acceptable carrier, such as water, to regenerate a liposomal suspension.

In the case of water-insoluble compounds, a pharmaceutical composition can be prepared containing the water-insoluble compound, such as for example, in an aqueous base emulsion. In such an instance, the composition will contain a sufficient amount of pharmaceutically acceptable emulsifying agent to emulsify the desired amount of the compound. Particularly useful emulsifying agents include phosphatidyl cholines and lecithin.

In particular embodiments, the compound is administered to the subject in a therapeutically effective amount, as that term is defined above. Dosages of pharmaceutically active compounds can be determined by methods known in the art, see, e.g., Remington's Pharmaceutical Sciences (Maack Publishing Co., Easton, Pa.). The therapeutically effective dosage of any specific compound will vary somewhat from compound to compound, and patient to patient, and will depend upon the condition of the patient and the route of delivery. As a general proposition, a dosage from about 0.1 to about 50 mg/kg will have therapeutic efficacy, with all weights being calculated based upon the weight of the compound, including the cases where a salt is employed. Toxicity concerns at the higher level can restrict intravenous dosages to a lower level such as up to about 10 mg/kg, with all weights being calculated based upon the weight of the compound, including the cases where a salt is employed. A dosage from about 10 mg/kg to about 50 mg/kg can be employed for oral administration. Typically, a dosage from about 0.5 mg/kg to 5 mg/kg can be employed for intramuscular injection. Particular dosages are about 1 μmol/kg to 50 μmol/kg, and more particularly to about 22 μmol/kg and to 33 μmol/kg of the compound for intravenous or oral administration, respectively.

In particular embodiments of the invention, more than one administration (e.g., two, three, four, or more administrations) can be employed over a variety of time intervals (e.g., hourly, daily, weekly, monthly, etc.) to achieve therapeutic effects.

The present invention finds use in veterinary and medical applications. Suitable subjects include both avians and mammals, with mammals being preferred. The term “avian” as used herein includes, but is not limited to, chickens, ducks, geese, quail, turkeys, and pheasants. The term “mammal” as used herein includes, but is not limited to, humans, bovines, ovines, caprines, equines, felines, canines, lagomorphs, etc. Human subjects include neonates, infants, juveniles, and adults. In other embodiments, the subject is an animal model of bone disease.

The G11 or F12 polynucleotides may further be used as chromosomal markers, i.e., to map the location of other genes. As another embodiment, the G11 or F12 polynucleotides can be used as genetic markers of diseases, e.g., bone and mineralization diseases. Linkage of these genes with diseases will facilitate gene typing whereby certain allelic variations within a population are linked to a disease, which can be used to identify genetically-susceptible individuals for that disease.

The finding that G11 and F12 gene products are involved in mineralization and interaction with TGF-β superfamily members point to these polypeptides as new drug targets for identifying compounds for treating bone diseases and conditions. Accordingly, in one aspect, the present invention provides methods of identifying a compound or compounds that bind to and/or modulate the activity of a G11 or F12 polypeptide. Any desired end-point can be detected, e.g., binding to the G11 or F12 polypeptide, gene or RNA, modulation of the activity of the G11 or F12 polypeptide, modulation of TGF-β superfamily pathways (e.g., bone morphogenic protein pathways), and/or interference with binding by a known regulator of a G11 or F12 gene or polypeptide. Methods of detecting the foregoing activities are known in the art and include the methods disclosed herein.

Any compound of interest can be screened according to the present invention. Suitable test compounds include organic and inorganic molecules. Suitable organic molecules can include but are not limited to polypeptides (including enzymes, antibodies and Fab′ fragments), carbohydrates, lipids, coenzymes, and nucleic acid molecules (including DNA, RNA and chimerics and analogs thereof) and nucleotides and nucleotide analogs. In particular embodiments, the compound is an antisense nucleic acid, an siRNA, or a ribozyme that inhibits production of G11 or F12 polypeptide.

Further, the methods of the invention can be practiced to screen a compound library, e.g., a combinatorial chemical compound library, a polypeptide library, a cDNA library, a library of antisense nucleic acids, and the like, or an arrayed collection of compounds such as polypeptide and nucleic acid arrays.

In one representative embodiment, the invention provides methods of screening test compounds to identify a test compound that binds to a G11 or F12 polypeptide or functional fragment thereof. Compounds that are identified as binding to the G11 or F12 polypeptide or functional fragment can be subject to further screening (e.g., for modulation of mineralization and or modulation of TGF-beta superfamily member pathways, and the like) using the methods described herein or other suitable techniques.

Also provided are methods of screening compounds to identify those that modulate the activity of a G11 or F12 polypeptide or functional fragment thereof. The term “modulate” is intended to refer to compounds that enhance (e.g., increase) or inhibit (e.g., reduce) the activity of the G11 or F12 polypeptide (or functional fragment). For example, the interaction of the G11 or F12 polypeptide or functional fragment with a binding partner can be evaluated. As another alternative, physical methods, such as NMR, can be used to assess biological function. Activity of the G11 or F12 polypeptide or functional fragment can be evaluated by any method known in the art, including the methods disclosed herein.

Compounds that are identified as modulators of G11 or F12 activity can optionally be further screened using the methods described herein (e.g., for binding to the G11 or F12 polypeptide or functional fragment thereof, gene or RNA, modulation of mineralization, and the like). The compound can directly interact with the G11 or F12 polypeptide or functional fragment, gene or mRNA and thereby modulate its activity. Alternatively, the compound can interact with any other polypeptide, nucleic acid or other molecule as long as the interaction results in a modulation of the activity of the G11 or F12 polypeptide or functional fragment.

As another aspect, the invention provides a method of identifying compounds that modulate mineralization. In one representative embodiment, the method comprises contacting a G11 or F12 polypeptide or functional fragment thereof with a test compound; and detecting whether the test compound binds to the G11 or F12 polypeptide or functional fragment and/or modulates the activity of the G11 or F12 polypeptide (or fragment). In another exemplary embodiment, the method comprises introducing a test compound into a cell that comprises the G11 or F12 polypeptide or functional fragment; and detecting whether the compound binds to the G11 or F12 polypeptide or functional fragment and/or modulates the activity of the G11 or F12 polypeptide or functional fragment in the cell. The G11 or F12 polypeptide can be endogenously produced in the cell. Alternatively or additionally, the cell can be modified to comprise an isolated polynucleotide encoding, and optionally overexpressing, the G11 or F12 polypeptide or functional fragment thereof.

The screening assay can be a cell-based or cell-free assay. Further, the G11 or F12 polypeptide (or functional fragment thereof) or polynucleotide can be free in solution, affixed to a solid support, expressed on a cell surface, or located within a cell.

With respect to cell-free binding assays, test compounds can be synthesized or otherwise affixed to a solid substrate, such as plastic pins, glass slides, plastic wells, and the like. For example, the test compounds can be immobilized utilizing conjugation of biotin and streptavidin by techniques well known in the art. The test compounds are contacted with the G11 or F12 polypeptide or functional fragment thereof and washed. Bound polypeptide can be detected using standard techniques in the art (e.g., by radioactive or fluorescence labeling of the G11 or F12 polypeptide or functional fragment, by ELISA methods, and the like).

Alternatively, the G11 or F12 target can be immobilized to a solid substrate and the test compounds contacted with the bound G11 or F12 polypeptide or functional fragment thereof. Identifying those test compounds that bind to and/or modulate the G11 or F12 polypeptide or functional fragment can be carried out with routine techniques. For example, the test compounds can be immobilized utilizing conjugation of biotin and streptavidin by techniques well known in the art. As another illustrative example, antibodies reactive with the G11 or F12 polypeptide or functional fragment can be bound to the wells of the plate, and the G11 or F12 polypeptide trapped in the wells by antibody conjugation. Preparations of test compounds can be incubated in the G11 or F12 polypeptide (or functional fragment)-presenting wells and the amount of complex trapped in the well can be quantitated.

In another representative embodiment, a fusion protein can be provided which comprises a domain that facilitates binding of the protein to a matrix. For example, glutathione-S-transferase fusion proteins can be adsorbed onto glutathione sepharose beads (Sigma Chemical, St. Louis, Mo.) or glutathione derivatized microtitre plates, which are then combined with cell lysates (e.g., ³⁵S-labeled) and the test compound, and the mixture incubated under conditions conducive to complex formation (e.g., at physiological conditions for salt and pH). Following incubation, the beads are washed to remove any unbound label, and the matrix immobilized and radiolabel detected directly, or in the supernatant after the complexes are dissociated. Alternatively, the complexes can be dissociated from the matrix, separated by SDS-PAGE, and the level of G11 or F12 polypeptide or functional fragment thereof found in the bead fraction quantitated from the gel using standard electrophoretic techniques.

Another technique for compound screening provides for high throughput screening of compounds having suitable binding affinity to the polypeptide of interest, as described in published PCT application WO84/03564. In this method, a large number of different small test compounds are synthesized on a solid substrate, such as plastic pins or some other surface. The test compounds are reacted with the G11 or F12 polypeptide or functional fragment thereof and washed. Bound polypeptide is then detected by methods well known in the art. Purified G11 or F12 polypeptide or a functional fragment can also be coated directly onto plates for use in the aforementioned drug screening techniques. Alternatively, non-neutralizing antibodies can be used to capture the peptide and immobilize it on a solid support.

With respect to cell-based assays, any suitable cell can be used, including bacteria, yeast, insect cells (e.g., with a baculovirus expression system), avian cells, mammalian cells, or plant cells. In exemplary embodiments, the assay is carried out in a cell line that naturally expresses the G11 or F12 gene or produces the polypeptide, e.g., osteoblasts or pre-osteoblasts such as MC3T3-E1 cells. Further, in other embodiments, it is desirable to use nontransformed cells (e.g., primary cells) as transformation may alter the function of the polypeptide.

The screening assay can be used to detect compounds that bind to or modulate the activity of the native G11 or F12 polypeptide (e.g., polypeptide that is normally produced by the cell). Alternatively, the cell can be modified to express (e.g., overexpress) a recombinant G11 or F12 polypeptide or functional fragment thereof. According to this embodiment, the cell can be transiently or stably transformed with a polynucleotide encoding the G11 or F12 polypeptide or functional fragment, but is preferably stably transformed, for example, by stable integration into the genome of the organism or by expression from a stably maintained episome (e.g., Epstein Barr Virus derived episomes).

In a cell-based assay, the compound to be screened can interact directly with the G11 or F12 polypeptide or functional fragment thereof (i.e., bind to it) and modulate the activity thereof. Alternatively, the compound can be one that modulates G11 or F12 polypeptide activity (or the activity of a functional fragment) at the nucleic acid level. To illustrate, the compound can modulate transcription of the G11 or F12 gene (or transgene), modulate the accumulation of G11 or F12 mRNA (e.g., by affecting the rate of transcription and/or turnover of the mRNA), and/or modulate the rate and/or amount of translation of the G11 or F12 mRNA transcript.

As a further type of cell-based binding assay, the G11 or F12 polypeptide or functional fragment thereof can be used as a “bait protein” in a two-hybrid or three-hybrid assay (see, e.g., U.S. Pat. No. 5,283,317; Zervos et al., Cell 72:223 (1993); Madura et al., J. Biol. Chem. 268:12046 (1993); Bartel et al., Biotechniques 14:920 (1993); Iwabuchi et al., Oncogene 8:1693 (1993); and PCT publication WO94/10300), to identify other polypeptides that bind to or interact with the G11 or F12 polypeptide or functional fragment thereof.

The two-hybrid system is based on the modular nature of most transcription factors, which consist of separable DNA-binding and activation domains. Briefly, the assay utilizes two different DNA constructs. In one construct, the polynucleotide that encodes the G11 or F12 polypeptide or functional fragment thereof is fused to a nucleic acid encoding the DNA binding domain of a known transcription factor (e.g., GAL-4). In the other construct, a DNA sequence, optionally from a library of DNA sequences, that encodes an unidentified protein (“prey” or “sample”) is fused to a nucleic acid that codes for the activation domain of the known transcription factor. If the “bait” and the “prey” proteins are able to interact in vivo, forming a complex, the DNA-binding and activation domains of the transcription factor are brought into close proximity. This proximity allows transcription of a reporter sequence (e.g., LacZ), which is operably linked to a transcriptional regulatory site responsive to the transcription factor. Expression of the reporter can be detected and cell colonies containing the functional transcription factor can be isolated and used to obtain the nucleic acid encoding the polypeptide that exhibited binding to the G11 or F12 polypeptide or functional fragment.

As another cell-based assay, the invention provides a method of screening a compound for modulation of mineralization. In particular embodiments, the cell comprises an isolated polynucleotide encoding the G11 or F12 polypeptide or functional fragment thereof. According to this embodiment, it is preferred that the isolated polynucleotide encoding the G11 or F12 polypeptide or functional fragment is stably incorporated into the cell (i.e., by stable integration into the genome of the organism or by expression from a stably maintained episome such as Epstein Barr Virus derived episomes).

Screening assays can also be carried out in vivo in animals. Thus, as still a further aspect, the invention provides a transgenic non-human animal comprising an isolated polynucleotide encoding a G11 or F12 polypeptide or functional fragment thereof, which can be produced according to methods well-known in the art. The transgenic non-human animal can be from any species, including avians and non-human mammals. According to this aspect of the invention, suitable non-human mammals include mice, rats, rabbits, guinea pigs, goats, sheep, pigs, and cattle. Suitable avians include chickens, ducks, geese, quail, turkeys, and pheasants.

The polynucleotide encoding the G11 or F12 polypeptide or functional fragment can be stably incorporated into cells within the transgenic animal (typically, by stable integration into the genome or by stably maintained episomal constructs). It is not necessary that every cell contain the transgene, and the animal can be a chimera of modified and unmodified cells, as long as a sufficient number of cells comprise and express the polynucleotide encoding the G11 or F12 polypeptide or functional fragment so that the animal is a useful screening tool.

Exemplary methods of using the transgenic non-human animals of the invention for in vivo screening of compounds that modulate mineralization, bone formation, bone loss, and/or the activity of a G11 or F12 polypeptide comprise administering a test compound to a transgenic non-human animal (e.g., a mammal such as a mouse) comprising an isolated polynucleotide encoding a G11 or F12 polypeptide or functional fragment thereof stably incorporated into the genome and detecting whether the test compound modulates mineralization, bone formation, bone loss, and/or G11 or F12 polypeptide activity (or the activity of a functional fragment).

It is known in the art how to measure these responses in vivo. Illustrative approaches include observation of changes that can be studied by gross examination (e.g., formation of mineralized nodules), histopathology (e.g., cell proliferation, cell differentiation), cell markers, and enzymatic activity (e.g., alkaline phosphatase).

Methods of making transgenic animals are known in the art. DNA or RNA constructs can be introduced into the germ line of an avian or mammal to make a transgenic animal. For example, one or several copies of the construct can be incorporated into the genome of an embryo by standard transgenic techniques.

In an exemplary embodiment, a transgenic non-human animal is produced by introducing a transgene into the germ line of the non-human animal. Transgenes can be introduced into embryonal target cells at various developmental stages. Different methods are used depending on the stage of development of the embryonal target cell. The specific line(s) of any animal used should, if possible, be selected for general good health, good embryo yields, good pronuclear visibility in the embryo, and good reproductive fitness.

Introduction of the transgene into the embryo can be accomplished by any of a variety of means known in the art such as microinjection, electroporation, lipofection, or a viral vector. For example, the transgene can be introduced into a mammal by microinjection of the construct into the pronuclei of the fertilized mammalian egg(s) to cause one or more copies of the construct to be retained in the cells of the developing mammal(s). Following introduction of the transgene construct into the fertilized egg, the egg can be incubated in vitro for varying amounts of time, or reimplanted into the surrogate host, or both. One common method is to incubate the embryos in vitro for about 1-7 days, depending on the species, and then reimplant them into the surrogate host.

The progeny of the transgenically manipulated embryos can be tested for the presence of the construct by Southern blot analysis of a segment of tissue. An embryo having one or more copies of the exogenous cloned construct stably integrated into the genome can be used to establish a permanent transgenic animal line.

Transgenically altered animals can be assayed after birth for the incorporation of the construct into the genome of the offspring. This can be done by hybridizing a probe corresponding to the polynucleotide sequence coding for the polypeptide or a segment thereof onto chromosomal material from the progeny. Those progeny found to contain at least one copy of the construct in their genome are grown to maturity.

Methods of producing transgenic avians are also known in the art, see, e.g., U.S. Pat. No. 5,162,215.

In particular embodiments, to create an animal model in which the activity or expression of a G11 or F12 polypeptide is decreased, it is desirable to inactivate, replace or knock-out the endogenous G11 or F12 gene by homologous recombination with a transgene using embryonic stem cells. In this context, a transgene is meant to refer to heterologous nucleic acid that upon insertion within or adjacent to the G11 or F12 gene results in a decrease or inactivation of G11 or F12 gene expression or G11 or F12 polypeptide amount or activity.

A knock-out of a G11 or F12 gene means an alteration in the sequence of a G11 or F12 gene that results in a decrease of function of the G11 or F12 gene, preferably such that the G11 or F12 gene expression or G11 or F12 polypeptide amount or activity is undetectable or insignificant. Knock-outs as used herein also include conditional knock-outs, where alteration of the G11 or F12 gene can occur upon, for example, exposure of the animal to a substance that promotes G11 or F12 gene alteration (e.g., tetracycline or ecdysone), introduction of an enzyme that promotes recombination at a G11 or F12 gene site (e.g., Cre in the Cre-lox system), or other method for directing the G11 or F12 gene alteration postnatally. Knock-out animals may be prepared using methods known to those of skill in the art. See, for example, Hogan, et al. (1986) Manipulating the Mouse Embryo: A Laboratory Manual, Cold Spring Harbor Laboratory Press, Cold Spring Harbor, N.Y.

A knock-out construct is a nucleic acid sequence, such as a DNA or RNA construct, which, when introduced into a cell, results in suppression (partial or complete) of expression of a polypeptide encoded by endogenous DNA in the cell. A knock-out construct as used herein may include a construct containing a first fragment from the 5′ end of the G11 or F12 gene, a second fragment from the 3′ end of the G11 or F12 gene and a DNA fragment encoding a selectable marker positioned between the first and second G11 or F12 fragments. It should be understood by the skilled artisan that any suitable 5′ and 3′ fragments of a G11 or F12 gene may be used as long as the expression of the corresponding G11 or F12 gene is partially or completely suppressed by insertion of the transgene. Suitable selectable markers include, but are not limited to, neomycin, puromycin and hygromycin. In addition, the construct may contain a marker, such as diphtheria toxin A or thymidine kinase, for increasing the frequency of obtaining correctly targeted cells. Suitable vectors include, but are not limited to, pBLUESCRIPT, pBR322, and pGEM7.

Alternatively, a knock-out construct may contain RNA molecules such as antisense RNA, siRNA, and the like to decrease the expression of a G11 or F12 gene. In particular embodiments, the siRNA molecules comprise SEQ ID NO:1, SEQ ID NO:3, SEQ ID NO: 5, SEQ ID NO:7, SEQ ID NO:9 and/or SEQ ID NO:11 or a fragment thereof. Typically, for stable expression the RNA molecule is placed under the control of a promoter. The promoter may be regulated, if deficiencies in the protein of interest may lead to a lethal phenotype, or the promoter may drive constitutive expression of the RNA molecule such that the gene of interest is silenced under all conditions of growth. While homologous recombination between the knock-out construct and the G11 or F12 gene of interest may not be necessary when using an RNA molecule to decrease G11 or F12 gene expression, it may be advantageous to target the knock-out construct to a particular location in the genome of the host organism so that unintended phenotypes are not generated by random insertion of the knock-out construct.

The knock-out construct may subsequently be incorporated into a viral or nonviral vector for delivery to the host animal or may be introduced into embryonic stem (ES) cells. ES cells are typically selected for their ability to integrate into and become part of the germ line of a developing embryo so as to create germ line transmission of the knock-out construct. Thus, any ES cell line that can do so is suitable for use herein. Suitable cell lines which may be used include, but are not limited to, the 129J ES cell line or the J1 ES cell line. The cells are cultured and prepared for DNA insertion using methods well-known to the skilled artisan (e.g., see Robertson (1987) In: Teratocarcinomas and Embryonic Stem Cells: A Practical Approach, E. J. Robertson, ed. IRL Press, Washington, D.C.; Bradley et al., Curr. Topics Develop. Biol. 20:357 (1986); Hogan et al., (1986) Manipulating the Mouse Embryo: A Laboratory Manual, Cold Spring Harbor Laboratory Press, Cold Spring Harbor, N.Y.).

Insertion of the knock-out construct into the ES cells may be accomplished using a variety of methods well-known in the art, including, for example, electroporation, microinjection, and calcium phosphate treatment. For insertion of the DNA or RNA sequence, the knock-out construct nucleic acids are added to the ES cells under appropriate conditions for the insertion method chosen. If the cells are to be electroporated, the ES cells and construct nucleic acids are exposed to an electric pulse using an electroporation machine (electroporator) and following the manufacturer's guidelines for use. After electroporation, the cells are allowed to recover under suitable incubation conditions. The cells are then screened for the presence of the knockout construct.

Each knock-out construct to be introduced into the cell is first typically linearized if the knock-out construct has been inserted into a vector. Linearization is accomplished by digesting the knock-out construct with a suitable restriction endonuclease selected to cut only within the vector sequence and not within the knock-out construct sequence.

Screening for cells which contain the knock-out construct (homologous recombinants) may be done using a variety of methods. For example, as described herein, cells can be processed as needed to render DNA in them available for hybridization with a nucleic acid probe designed to hybridize only to cells containing the construct. For example, cellular DNA can be probed with ³²P-labeled DNA which locates outside the targeting fragment. This technique can be used to identify those cells with proper integration of the knock-out construct. The DNA can be extracted from the cells using standard methods (e.g., see, Sambrook et al., Molecular Cloning: A Laboratory Manual 2nd Ed. (Cold Spring Harbor, N.Y., 1989)). The DNA may then be analyzed by Southern blot with a probe or probes designed to hybridize in a specific pattern to genomic DNA digested with one or more particular restriction enzymes.

Once appropriate ES cells are identified, they are introduced into an embryo using standard methods. They can be introduced using microinjection, for example. Embryos at the proper stage of development for integration of the ES cell to occur are obtained, such as by perfusion of the uterus of pregnant females. For example, mouse embryos at 3-4 days development can be obtained and injected with ES cells using a micropipet. After introduction of the ES cell into the embryo, the embryo is introduced into the uterus of a pseudopregnant female mouse. The stage of the pseudopregnancy is selected to enhance the chance of successful implantation. In mice, 2-3 days pseudopregnant females are appropriate.

Germline transmission of the knockout construct may be determined using standard methods. Offspring resulting from implantation of embryos containing the ES cells described above are screened for the presence of the desired alteration (e.g., G11 or F12 knock-out). This may be done, for example, by obtaining DNA from offspring (e.g., tail DNA) to assess for the knock-out construct, using known methods (e.g., Southern analysis, dot blot analysis, PCR analysis). See, for example, Sambrook et al., Molecular Cloning: A Laboratory Manual 2nd Ed. (Cold Spring Harbor, N.Y., 1989). Offspring identified as chimeras may be crossed with one another to produce homozygous knock-out animals.

Mice are often used as animal models because they are easy to house, relatively inexpensive, and easy to breed. However, other knock-out animals may also be made in accordance with the present invention such as, but not limited to, monkeys, cattle, sheep, pigs, goats, horses, dogs, cats, guinea pigs, rabbits and rats. Accordingly, appropriate vectors and promoters well-known in the art may be selected and used to generate a transgenic animal deficient in G11 or F12 expression.

The present invention is more particularly described in the following examples that are intended as illustrative only since numerous modifications and variations therein will be apparent to those skilled in the art.

### EXAMPLE 1

### Identification and Characterization of G11 and F12

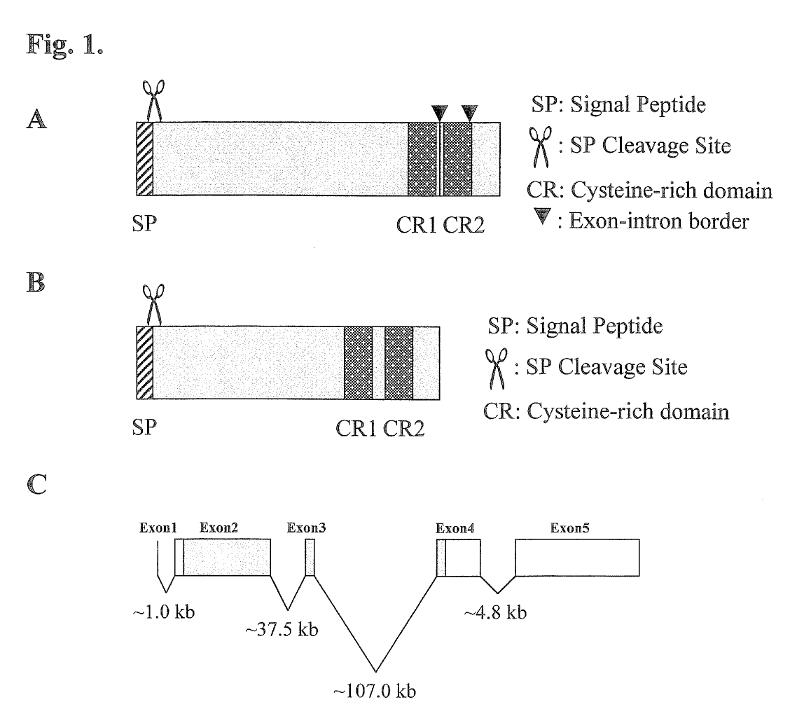

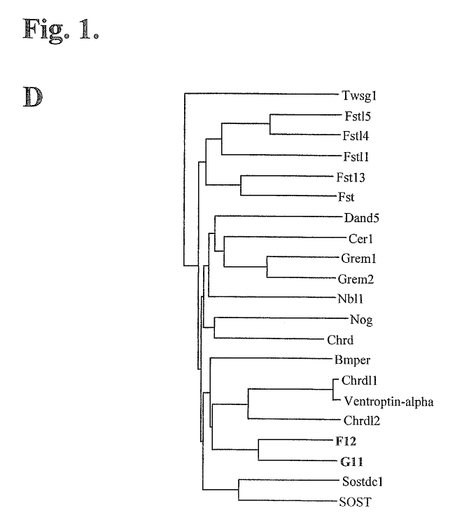

To identify new cysteine knot proteins (CKP), the mouse ortholog of the chordin sequence (a well known BMP binding protein (GenBank accession number; NP_{—}034023)) was obtained and a BLAST search was performed in the mouse genome (www.ncbi.nlm.nih.gov/genome/seq/MmBlast.html) with some modifications on expect, filter, descriptions and alignments parameters. Two hundred and twenty five candidate sequences were obtained from the search, and the subcellular localization of each protein was predicted using the PSORT II program (psort.nibb.acjp/form2.html). This computational screening identified two extracellular molecules that have a signal peptide sequence but lack a transmembrane domain. The genes were originally cloned as RIKEN cDNA A930041G11 and RIKEN cDNA A830006F12 by the RIKEN mouse Genome Exploration Research Group. Neither gene had ever been characterized and are referred to herein as “G11” and “F12.” The predicted amino acid sequences of G11 and F12 have characteristic cysteine knot sequences commonly found in CKP family (FIG. 1A, B).

After the computational search, the cDNA sequences containing the coding region of mouse G11, mouse F12, and human F12 were isolated by reverse transcription-polymerase chain reaction (RT-PCR) using Proofstart DNA polymerase (QIAGEN). A cDNA library derived from MC3T3-E1 preosteoblastic cells (subclone 4, ATCC # CRL-2593), a well-characterized, non-transformed preosteoblastic cell line, was used as a template for RT-PCR. A cDNA library derived from human brain was purchased from Clontech and used for a template when human F12 was cloned. The PCR conditions were as follows: 15 min at 95° C.; 30 s at 95° C., 1 min at 60° C., 1 min at 72° C. for cycles. The amplified PCR products were separated on a 1.2% TAE-agarose gel, stained with ethidium bromide, and photographed under UV light. The PCR products were then ligated into the pcDNA3.1-V5/His-TOPO mammalian expression vector (Invitrogen), transformed into bacterial competent cells. The cDNA was then purified using a Miniprep kit (Qiagen) and the plasmids were analyzed for the insertion and their orientation by restriction enzymes. They were sequenced at the UNC-CH DNA Sequencing Facility (University of North Carolina, Chapel Hill, N.C.), and the DNA sequences of PCR products were 100% identical to the ones deposited in the NCBI database, verifying the presence of the transcript in MC3T3-E1 cells and brain. Splicing variants of F12 were identified through the gene cloning: there are 2 isoforms in human and 3 in mouse. The sequences identified were deposited in GenBank under the following accession numbers; mouse G11: DQ421811, human F12-1: EU541473, human F12-2: EF552207, mouse F12-1: EF552208, mouse F12-2: EF552209, mouse F12-3: EF552210. Mouse G11 and mouse F12-1 were used in the following studies.

Primer sequences for amplifying the open reading frame of mouse G11 and F12 were designed, cDNA sequences containing the coding region of G11 and F12 were isolated in the same manner as described above, and the plasmid containing G11 or F12 cDNA was successfully obtained (pcDNA3.1-G11-V5/His or pcDNA3.1-F12-V5/His). G11 or F12 cDNA was also ligated into the pcDNA3 vector (Invitrogen) with hemagglutinin (HA) tag (pcDNA3-G11-HA or pcDNA3-F12-HA) and purified in the same manner as above.

The G11 gene consists of 5 exons (FIG. 1C) and is localized at mouse chromosome locus 11 and human chromosome locus 7p12.3-p12.2 based on the NCBI database. The F12 gene is localized at mouse chromosome locus 1 and human chromosome locus 2q34-q35 based on the NCBI database. In order to confirm that G11 and F12 are novel CKP members, the protein sequences of the other known CKPs in mouse were obtained from the public database deposited as NCBI reference sequences, and phylogenetic tree analysis of G11 and F12 together with the other known CKPs was performed using Clustal W server (www.ch.embnet.org/software/ClustalW.html) and the Clustal X program. The results showed that G11 and F12 were categorized in a new subgroup and that they belong to a new class of the CKP family, thus, representing novel CKP family members (FIG. 1D).

### EXAMPLE 2

### Expression of G11 and F12

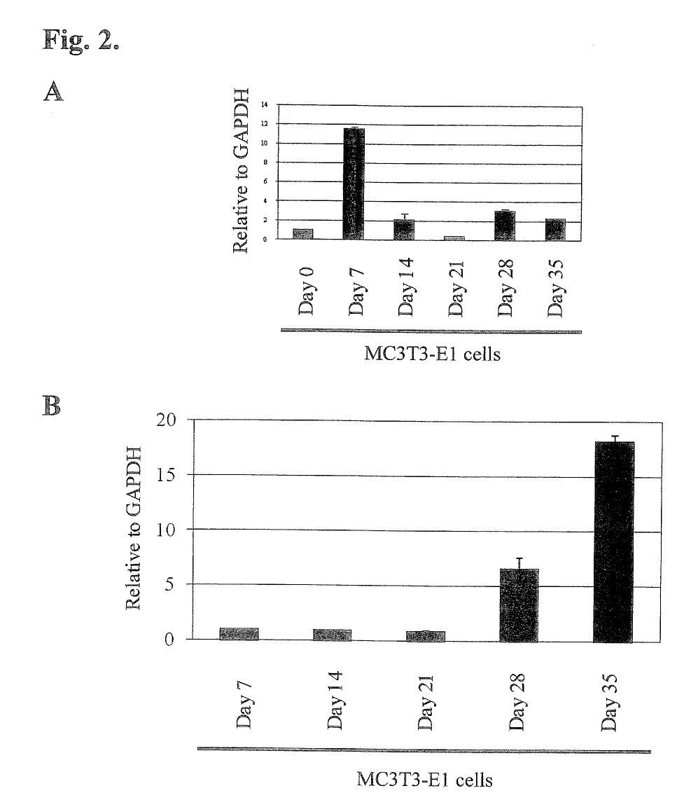

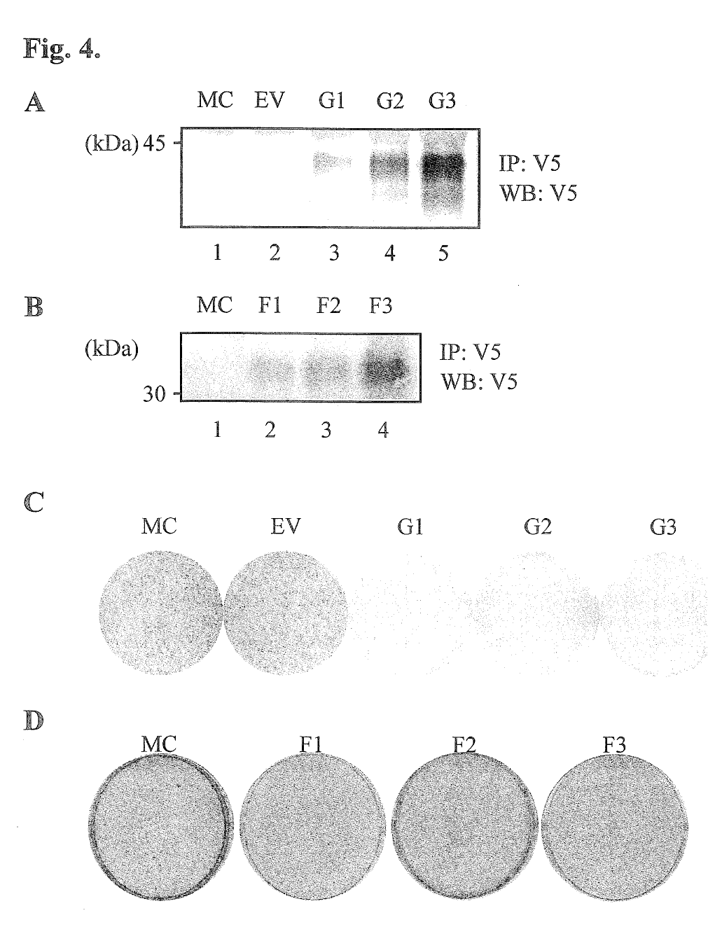

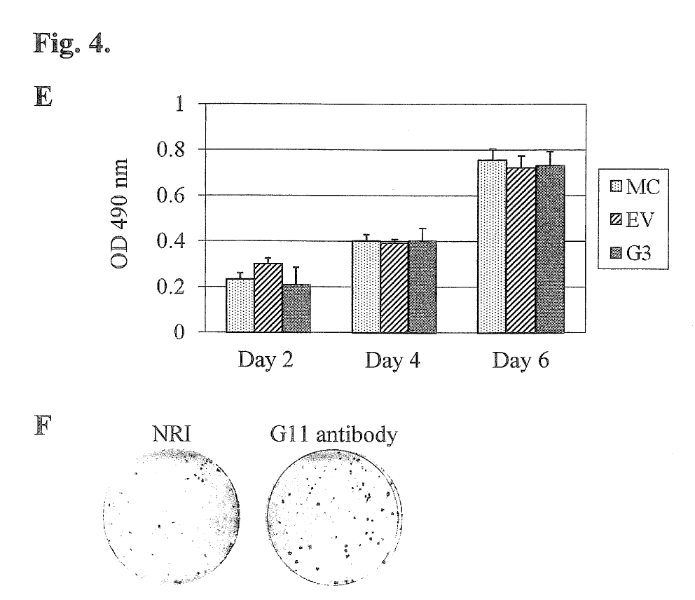

In order to investigate the expression pattern of G11/F12 genes, real time PCR was performed using the cDNA derived from MC3T3-E1 preosteoblastic cells. MC3T3-E1 cells (subclone 4) were maintained in α-minimum essential medium (α-MEM) containing 10% fetal bovine serum (FBS) and plated on 35 mm dishes (Falcon) at a density of 2×10⁵ cells/dish. After reaching confluence, the medium was replaced with medium supplemented with 50 μg/ml ascorbic acid and 1 mM β-glycerophosphate (mineralization medium) (this time point is referred to as day 0), and maintained for up to 35 days. Total RNA was extracted with TRIzol reagent solution (Invitrogen) at the end of each week. Two μg of the total RNA extract was used for reverse transcription (RT) using the Omniscript RT Kit (Qiagen) according to the manufacturer's protocol. Real time PCR was performed in triplicate using the specific primers-probe for G11 (ABI assay number: Mm00621766_m1), F12 (ABI assay number: Mm01260094_m1) or glyceraldehyde-3-phosphate dehydrogenase (GAPDH, ABI assay number: 4308313), and the expression levels were analyzed by the ABI Prism 7000 Sequence detection system (Applied Biosystems). The mean fold changes in the expression of G11 or F12 relative to that of GAPDH were calculated using the values obtained from the cDNA at day 0 or day 7 as a calibrator by means of 2^{-ΔC}_{T} method. The results demonstrated that the expression of G11 in MC3T3-E1 cells at day 7 was the highest (˜12 fold of that at day 0, FIG. 2A) during matrix mineralization. F12 mRNA expression pattern in MC3T3-E1 cells was quantitatively analyzed the and the expression level was markedly increased at day 28 and 35, i.e., ˜7 fold at day 28 and ˜18 fold at day 35 compared to that of day 7 (FIG. 2B). In another set of experiments, the mineralized nodules formed in the cultures (see FIG. 4C, 4D) were analyzed by Alizarin Red S (Sigma) staining and the initial mineralized nodule formation was detected at day 21˜28. Thus, the results suggest that the expression of these new genes is potentially associated with matrix mineralization.

### EXAMPLE 3

### Generation of G11 Antibody

In order to characterize the G11 protein, a synthetic peptide corresponding to amino acid positions 49-64 of mouse G11 (SEQ ID NO:2) was generated (i.e., E⁴⁹HASRDSPGRVSELGR⁶⁴, SEQ ID NO:13) and an affinity purified-polyclonal G11 antibody was generated by immunizing rabbits with the peptide (Bethyl laboratories Inc.). To characterize the anti-G11 antibody, G11 protein was generated and purified as follows. 293 cells (Clontech) were transfected with the pcDNA3.1-G11-V5/His construct using FuGENE6 transfection reagent (Roche Diagnostics) according to the manufacturer's instructions. The cells were cultured in Dulbecco's modified Eagle's medium (DMEM, Invitrogen) containing 10% FBS and 400 μg/ml G418 (Gibco) for 3-4 weeks, and clones derived from G418-resistant cells were isolated by cloning rings and further grown under the same conditions. Equal numbers of cells in each clone were plated, cultured for 3 days, and cultured media were collected and immunoprecipitated (IP) with anti-V5 antibody (Invitrogen). After addition of Protein A-sepharose 4B conjugate (Zymed Laboratories), the samples were incubated for 30 min, and the beads were washed twice with lysis buffer containing 150 mM NaCl, 20 mM Tris-HCl pH 7.5, 10 mM EDTA, 1% Triton X-100, 1% deoxycholate, 1.5% aprotinin and 1 mM phenylmethylsulfonyl fluoride (PMSF). The conjugated proteins were then released from the beads by boiling in SDS sample buffer for 5 min, applied to 4-12% SDS-PAGE under reducing conditions, transferred onto a PVDF membrane and subjected to Western blot (WB) analysis with anti-V5 antibody. The immunoreactivity was visualized by alkaline phosphatase conjugate substrate kit (Bio-Rad). The clone which synthesized the highest level of G11-V5/His protein was further cultured for 6 days and the cultured medium was collected. G11-V5/His fusion protein was purified using a Ni-NTA agarose resin (Qiagen), and purified proteins were pooled, dialyzed against distilled water, lyophilized and dissolved in distilled water. The protein concentration was measured using a DC protein assay kit (Bio-Rad). The purity of the G11-V5/His protein was assessed by 4-12% SDS-PAGE stained with Coomassie Brilliant Blue R-250 (CBB, Bio-Rad) (>90% purity) and the purified G11 protein was kept at −20° C. until use.

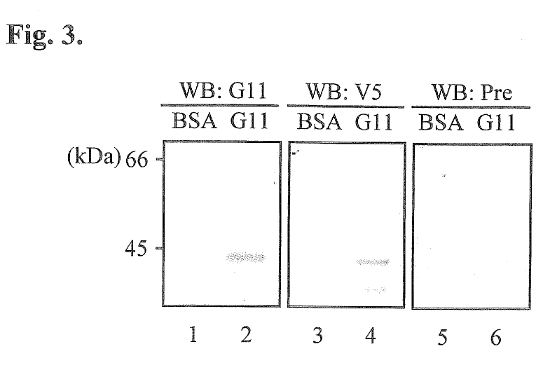

Equal amounts of purified G11-V5/His fusion protein (see above) and bovine serum albumin (BSA) (measured by a DC protein assay kit) were prepared and subjected to Western blot (WB) analysis with anti-G11 antibody. Preimmune serum was used to confirm the specificity of anti-G11 antibody. An immunoreactive band was detected when G11-V5/His protein, but not BSA, is present (FIG. 3, lanes 1 and 2) and the immunoreactivity of G11-V5/His fusion protein was verified using anti-V5 antibody (FIG. 3, lane 4). No immunoreactivity was observed and the specificity of anti-G11 antibody was thus confirmed when treated with preimmune serum (FIG. 3, lanes 5 and 6). The data clearly confirm the specificity of the generated anti-G11 antibody.

### EXAMPLE 4

### G11 and F12 Inhibit Matrix Mineralization

### Generation of MC3T3-E1 Preosteoblastic Cell Derived Clones Overexpressing G11 or F12 Protein

In order to obtain an insight into the biological roles of G11 and F12 in matrix mineralization, a “gain of function” approach was taken and MC3T3-E1 preosteoblastic cell derived clones that stably overexpress G11-V5/His protein (G clones) or F12-V5/His protein (F clones) were established. MC3T3-E1 cells were transfected with the pcDNA3.1-G11-V5/His or pcDNA3.1-F12-V5/His construct and maintained in α-MEM containing 10% FBS and 400 μg/ml G418 for up to 3-4 weeks. Stable cell clones were isolated by cloning rings and further cultured under the same conditions. MC3T3-E1 cells were also transfected with an empty pcDNA3.1-V5/His A vector (Invitrogen) and a clone (EV clone) was generated in the same manner. To verify that the G or F clones overexpress G11-V5/His or F12-V5/His protein, each of three clones (G1-G3 and F1-F3) and controls (MC3T3-E1 cells and/or EV clone) were seeded on 10 cm dishes at a density of 1×10⁶ cells/dish and cultured for 2 days. The cultured medium was then collected, and IP-WB analysis was performed with anti-V5 antibody as described above. The immunoreactive G11-V5/His or F12-V5/His bands were seen in G1-G3 (FIG. 4A, lanes 3-5) or F1-F3 clones (FIG. 4B, lanes 2-4), confirming that clones overexpressing G11-V5/His or F12-V5/His protein were successfully established.

### In Vitro Mineralization Assay

The three clones of G11 and F12 (G1-G3 and F1-F3) were then subjected to an in vitro mineralization assay. The controls and clones were maintained in α-MEM containing 10% FBS and plated on 35 mm dishes at a density of 2×10⁵ cells/dish. The medium was then changed to the mineralization medium as described above and cells/clones were cultured for up to 35 days. The cell/matrix layers were washed with PBS, fixed with 100% methanol and stained with 1% Alizarin Red S (Sigma) to visualize the mineralized nodule formation. At day 35, when the mineralized nodules were well formed in controls, the nodule formation was not detected in any G clones (FIG. 4C). In an independent experiment, MC3T3-E1 cells and F clones were cultured in the same manner as described above. When the initial mineralized nodules were evident in MC3T3-E1 cells at day 28, mineralized nodules in F clones were not detected (FIG. 4D). These results indicate that the onset and growth of mineralization were inhibited in G and F clones when compared with the controls.

### Effect of G11 on Cell Proliferation

Since inhibition of matrix mineralization can be due to the effects of G11 on cell proliferation, a proliferation assay was performed. The control cells (MC and EV) and G3 clone (which exhibited the highest expression of G11 among G clones) were plated in triplicate at a density of 1×10⁴ cells/well in a 96-well plate, cultured for up to 6 days until confluence and subjected to MTS cell proliferation assay (CellTier 96®, Promega) at day 2, 4 and 6 according to the manufacturer's protocol. The amount of formazan compound produced by metabolically active cells was measured by absorbance at 490 nm and the results demonstrated that there was no difference in the rate of cell proliferation between the G3 clone and controls (FIG. 4E).

### Effect of G1 Antibody on Matrix Mineralization

Since overexpression of G11 inhibits matrix mineralization, the effect of G11 antibody addition into MC3T3-E1 cell cultures on matrix mineralization was investigated. MC3T3-E1 cells were maintained in α-MEM containing 10% FBS and plated on 35 mm dishes at a density of 2×10⁵ cells/dish. On the following day, 0.5 μg/ml of normal rabbit immunoglobulin (NRI, control) or anti-G11 antibody was added into the cell cultures and further maintained. The medium was changed twice a week with an addition of NRI or anti-G11 antibody. The in vitro mineralization assay was performed in the same manner as described above at 17 days in culture. The cell/matrix layers were washed with PBS, fixed with 100% methanol and stained with 1% Alizarin Red S (Sigma) to visualize the mineralized nodule formation. At day 17, when the initial mineralized nodules were not formed in cells treated with NRI, nodule formation was clearly detected in cells treated with anti-G11 antibody (FIG. 4F), indicating that G11 antibody can accelerate the onset and growth of matrix mineralization.

### EXAMPLE 5

### Binding of G11 and F12 to TGF-β Superfamily Members

cDNA Cloning of TGF-β Superfamily Members (TGFs)

Mouse TGF-β1, TGF-β1 LAP region, TGF-β2, TGF-β3, BMP-2, BMP-4, BMP-6, BMP-7, inhibin-a, inhibin-ba, inhibin-bb and nodal cDNAs were isolated by RT-PCR using Hotstar Taq polymerase (Qiagen). The PCR products were then ligated into pcDNA3.1-V5/His-TOPO mammalian expression vectors (Invitrogen) and transformed to bactria JM109 strain. cDNAs were purified, digested with restriction enzyme, sequenced at the UNC-CH DNA Sequencing Facility, and the plasmids harboring V5/His (pcDNA3.1-TGFs-V5/His)-tagged TGFs cDNAs were obtained.

### Binding of G11 and F12 to TGFs.

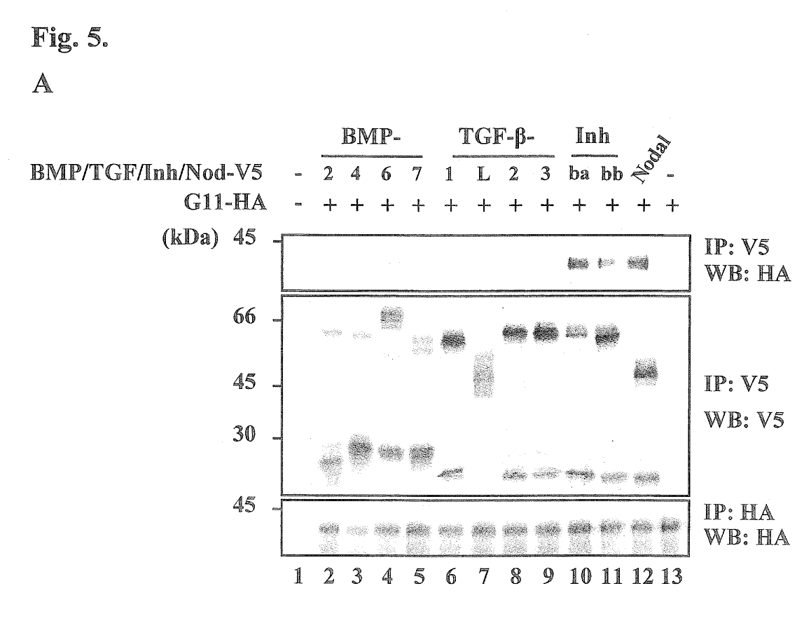

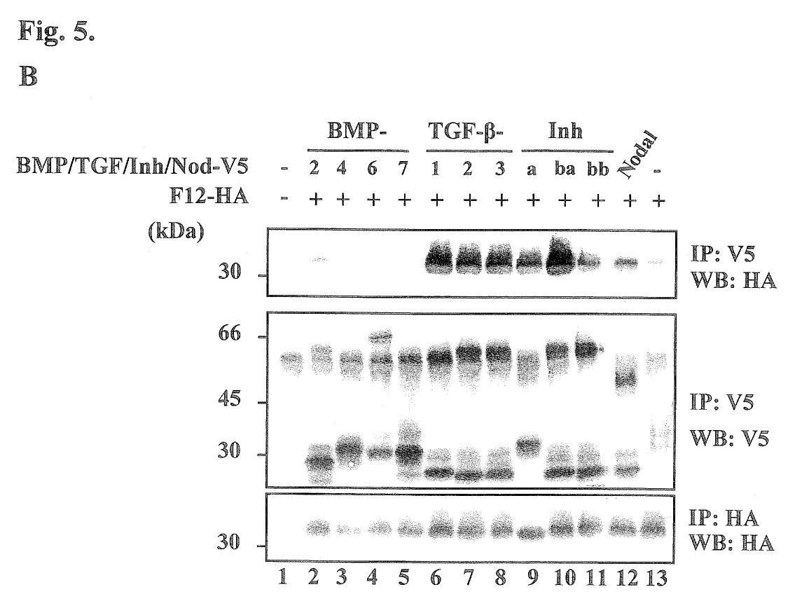

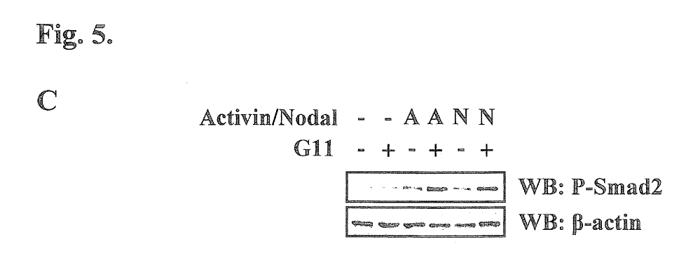

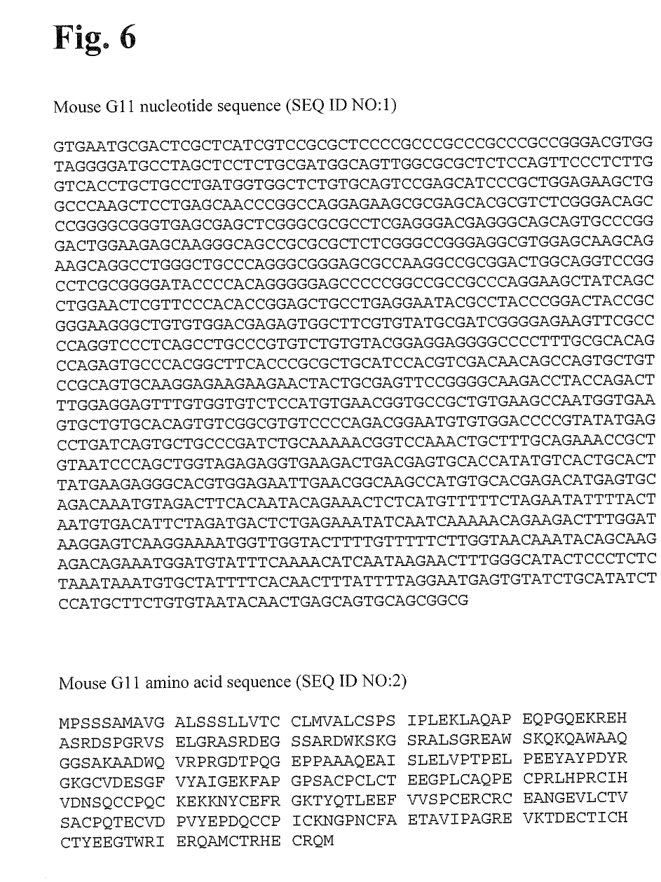

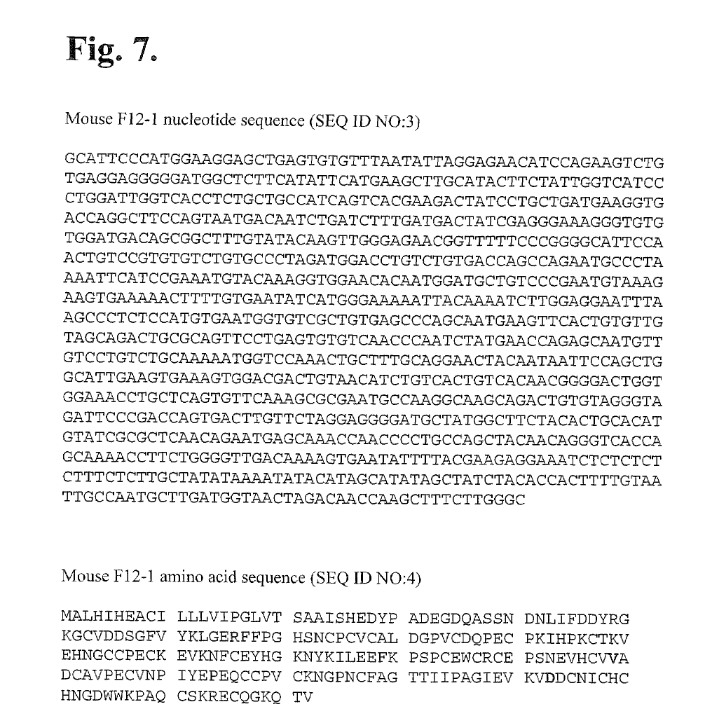

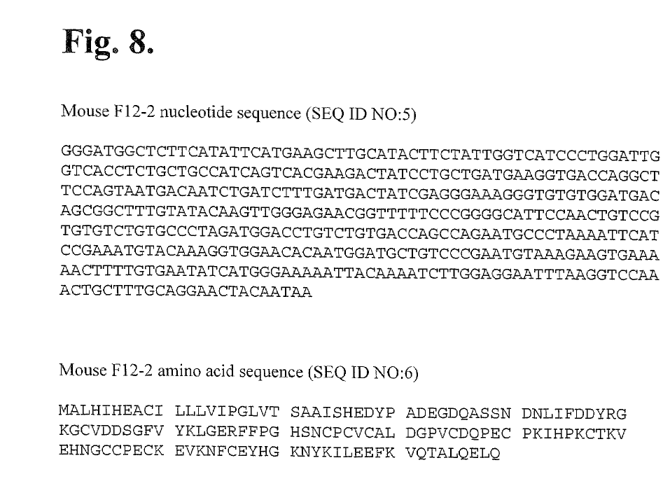

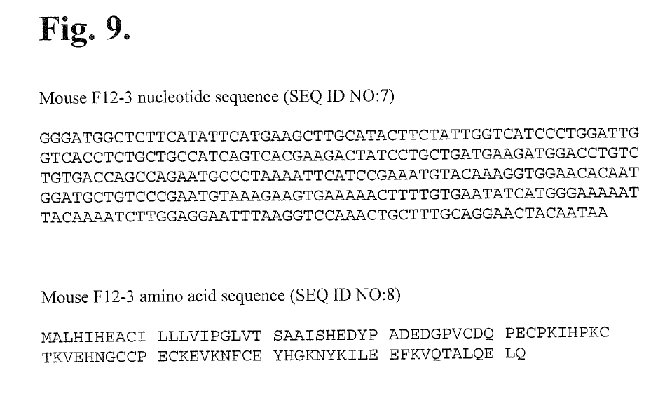

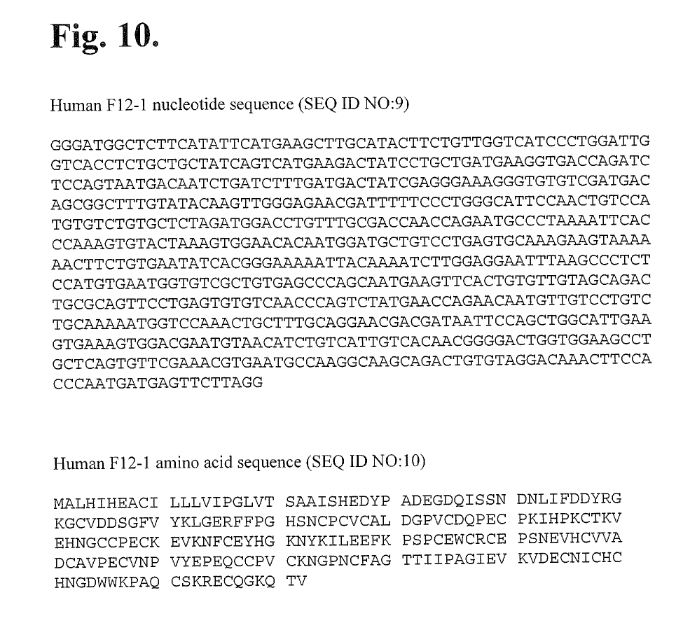

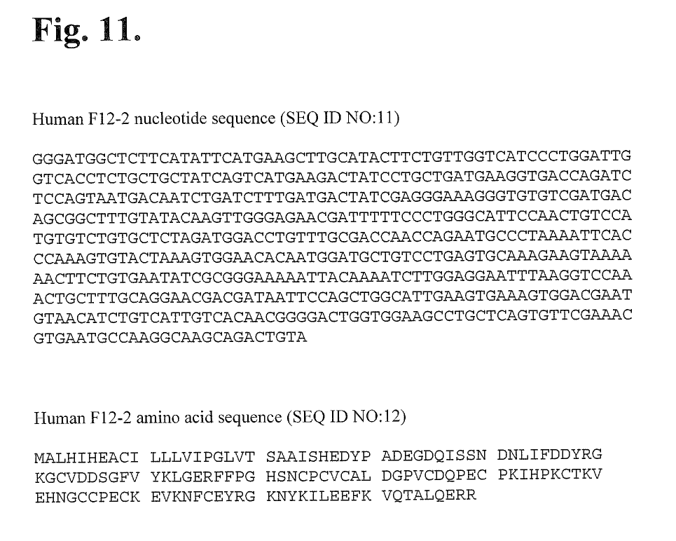

In order to investigate the potential binding between G11/F12 and TGFs, experiments were performed using an immunoprecipitation assay. The human embryonic kidney 293 cell line which is known to possess high transfection efficiency was used to transiently overexpress the tagged proteins. The cells were maintained in Dulbecco's Modified Eagle Media (DMEM) and plated on 6-well culture plates at a density of 3×10⁵ cells/well and transfected with 1.0 μg of pcDNA3.1-TGFs-V5/His, 1.0 μg of pcDNA3-G11-HA or 1.0 μg of pcDNA3-F12-HA, and/or 1.0 or 2.0 μg of empty pcDNA3.1-V5/His A vector using FuGENE6 transfection reagent (Roche Diagnostics) according to the manufacturer's protocol. Thus, total DNA amounts of each transfection were kept constant (2.0 μg). After the transfection, the cultured media were collected, immunoprecipitated (IP) with either anti-V5 (Invitrogen) or anti-HA antibody (Roche Diagnostics). The samples were then incubated with protein A-sepharose 4B conjugate beads (Zymed Laboratories) for 30 min and the beads were washed twice with lysis buffer. The conjugated protein complexes were then released from the beads by boiling for 5 min in SDS sample buffer, applied to 4-12% SDS-PAGE under reducing condition, transferred onto a PVDF membrane and subjected to Western blot (WB) analysis with either anti-V5 antibody or anti-HA antibody. The immunoreactivity was visualized by alkaline phosphatase conjugate substrate kit. When IP and WB were performed with either V5 or HA alone, V5-tagged TGFs or HA-tagged G11 (FIG. 5A) or F12 (FIG. 5B) was immunostained at the expected molecular weight ranges demonstrating that the transfection was successful. When IP was performed with anti-HA antibody followed by WB analysis with anti-V5 antibody to identify G11-binding TGFs partners (FIG. 5A), distinct immunoreactive bands were observed for the combination of G11-HA and inhibin ba-V5 (top panel, lane 10), G11-HA and inhibin bb-V5 (top panel, lane 11) and G11-HA and nodal-V5 (top panel, lane 12). The data clearly indicate that G11 specifically binds to these three TGFs members (FIG. 5A).

When IP was performed with anti-HA antibody followed by WB analysis with anti-V5 antibody to identify F12-binding TGFs partners (FIG. 5B), distinct immunoreactive bands were observed for the combination of F12-HA and all TGF-β isoforms (top panel, lanes 6-8), F12-HA and inhibin a-V5 (top panel, lane 9), F12-HA and inhibin ba-V5 (top panel, lane 10), F12-HA and inhibin bb-V5 (top panel, lane 11) and F12-HA and nodal-V5 (top panel, lane 12). The data clearly indicate that F12 binds to certain TGFs members in a slightly different from G11.

### Effect of G11 Protein on Activin/Nodal Signaling.

MC3T3-E1 cells were maintained in α-MEM containing 10% FBS and plated on 35 mm dishes at a density of 2×10⁵ cells/dish. On the following day, cells were treated with 200 ng/ml of activin A (R&D systems), 500 ng/ml of nodal (R&D systems) and 7.5 μg/ml of G11-V5/His protein for 30 min. Cell lysates were prepared and subjected to WB analysis using phospho-Smad2 (Cell Signaling) and anti-β-actin (Sigma) antibodies. The level of Smad2 phosphorylation was increased either by activin A (A) or nodal (N) addition. When G11 protein was treated together with activin A or nodal, the level of Smad2 phosphorylation was enhanced. The data indicate that G11 enhances Smad2 dependent signaling, possibly through association with inhibins and nodal.

The foregoing is illustrative of the present invention, and is not to be construed as limiting thereof. The invention is defined by the following claims, with equivalents of the claims to be included therein.

That which is claimed is:

1. An isolated polynucleotide selected from the group consisting of:
(a) a polynucleotide comprising a nucleotide sequence at least 70% identical to a nucleotide sequence selected from the group consisting of SEQ ID NOS:1, 3, 5, 7, 9, and 11, and encoding a functional polypeptide;
(b) a polynucleotide that hybridizes to a nucleotide sequence selected from the group consisting of SEQ ID NOS:1, 3, 5, 7, 9, and 11 under stringent hybridization conditions and encodes a functional polypeptide;
(c) a polynucleotide encoding a functional polypeptide comprising an amino acid sequence at least 70% identical to an amino acid sequence selected from the group consisting of SEQ ID NOS:2, 4, 6, 8, 10, and 12; and
(d) a functional fragment of any of (a) to (c).

2. The isolated polynucleotide of claim 1, wherein said isolated polynucleotide is selected from the group consisting of:
(a) a polynucleotide comprising a nucleotide sequence selected from the group consisting of SEQ ID NOS:1, 3, 5, 7, 9, and 11 or a fragment thereof that encodes a functional polypeptide;
(b) a polynucleotide encoding a functional polypeptide comprising an amino acid sequence selected from the group consisting of SEQ ID NOS:2, 4, 6, 8, 10, and 12 or a functional fragment thereof; and
(c) a polynucleotide comprising a nucleotide sequence that differs from the nucleotide sequences of (a) or (b) above due to the degeneracy of the genetic code.

3. A vector comprising the isolated polynucleotide of claim 1.

4. A cell comprising the isolated polynucleotide of claim 1.

5. A cell comprising the vector of claim 3.

6. An isolated polypeptide encoded by the isolated polynucleotide of claim 1.

7. A cell comprising the isolated polypeptide of claim 6.

8. A fusion protein comprising the polypeptide of claims 6.

9. An isolated polynucleotide encoding the fusion protein of claim 8.

10. A recombinant method of producing a polypeptide, comprising culturing the cell of claim 5 under conditions such that said polypeptide is expressed and recovering said polypeptide.

11. An antibody that specifically binds to the polypeptide of claim 6.

12. An antisense oligonucleotide, ribozyme, or siRNA that specifically binds to the polynucleotide of claim 1.

13. A pharmaceutical composition comprising the polynucleotide of claim 1 and a pharmaceutically acceptable carrier.

14. A pharmaceutical composition comprising the polypeptide of claim 6 and a pharmaceutically acceptable carrier.

15. A pharmaceutical composition comprising the antibody of claim 11 and a pharmaceutically acceptable carrier.

16. A pharmaceutical composition comprising the antisense oligonucleotide, ribozyme, or siRNA of claim 12 and a pharmaceutically acceptable carrier.

17. A method of inhibiting mineralization in a cell, comprising delivering to said cell a polynucleotide of claim 1 or a polypeptide of claim 6.

18. A method of inhibiting bone formation in a subject, comprising delivering to said subject a polynucleotide of claim 1 or a polypeptide of claim 6.

19. A method of inhibiting bone loss in a subject, comprising delivering to said subject a compound that decreases the activity of the polypeptide of claim 6 in an amount effective to decrease the activity of the polypeptide in the cell.

20. The method of claim 19, wherein the compound is selected from the group consisting of an antisense oligonucleotide, a ribozyme, and a siRNA that targets a polynucleotide encoding the polypeptide.

21. The method of claim 19, wherein the compound is an antibody that binds to the polypeptide.

22. A method of identifying a compound that binds to the polypeptide of claim 6, comprising:
contacting the polypeptide with a test compound under conditions whereby binding between the polypeptide and the test compound can be detected; and
detecting binding between the polypeptide and the test compound.

23. A method of identifying a compound that modulates the activity of a polypeptide of claim 6, comprising:
contacting the polypeptide with a test compound under conditions whereby modulation of the activity of the polypeptide can be detected; and
detecting modulation of the activity of the polypeptide.

24. The method of claim 22 or 23, wherein the method is carried out in a cell comprising the polypeptide.

25. The method of claim 24, wherein the cell comprises an isolated polynucleotide comprising a nucleotide sequence encoding the polypeptide.

26. The method of claim 25, wherein the cell is stably transformed with the isolated polynucleotide.

27. The method of claim 22 or 23, wherein the method is carried out as a cell-free assay.

28. The method of claim 22 or 23, wherein the method is carried out in a transgenic non-human mammal comprising an isolated polynucleotide comprising a nucleotide sequence encoding the polypeptide.
